(19) United States
(12) Patent Application Publication
(10) Pub. No.: US 2026/0167734 A1
(43) Pub. Date: Jun. 18, 2026

(54) MONOCLONAL ANTIBODIES SPECIFIC FOR FAS LIGAND AND USES THEREOF

(71) Applicant: Providence Health & Services - Oregon, Portland, OR (US)

(72) Inventor: Matthew Taylor, Portland, OR (US)

(73) Assignee: Providence Health & Services - Oregon, Portland, OR (US)

(21) Appl. No.: 19/126,198

(22) PCT Filed: Oct. 30, 2023
(86) PCT No.: PCT/US2023/078201

§ 371 Date: Apr. 30, 2025

Related U.S. Application Data
(60) Provisional application No. 63/381,796, filed on Nov. 1, 2022.

(51) Int. Cl. C07K 16/28; A61K 40/31; A61K 40/42; A61K 47/68; A61P 35/00

(52) U.S. Cl. CPC C07K 16/2875; A61K 40/31; A61K 40/4215; A61K 47/6849; A61P 35/00; A61K 2239/13; A61K 2239/47; C07K 2317/24; C07K 2317/31; C07K 2317/52; C07K 2317/565

(57) ABSTRACT

Monoclonal antibodies that specifically bind to and block the function of Fas ligand (FasL) are described. The FasL-specific antibodies can be used for the development of therapeutics for the treatment of diseases, disorders and conditions associated with the Fas/FasL signaling pathway, such as cancer, sepsis, ischemia-reperfusion injury, and coronavirus disease 2019 (COVID-19).

### CROSS REFERENCE TO RELATED APPLICATIONS

This application claims the benefit of U.S. Provisional Application No. 63/381,796, filed Nov. 1, 2022, which is herein incorporated by reference in its entirety.

### FIELD

This disclosure concerns monoclonal antibodies that specifically bind Fas ligand (FasL) and methods of their use, such as for inhibiting Fas/FasL signaling in a subject.

### INCORPORATION OF ELECTRONIC SEQUENCE LISTING

The electronic sequence listing, submitted herewith as an XML file named 6727-109081-02.xml (24,326 bytes), created on Oct. 23, 2023, is herein incorporated by reference in its entirety.

### BACKGROUND

Fas ligand (FasL, also known as CD95L) is the ligand for the Fas (CD95) receptor. FasL binding to its receptor can induce either apoptotic or non-apoptotic signaling. Apoptotic signaling results in cell death of the Fas receptor-expressing cell. Non-apoptotic signaling through FasL/Fas receptor interactions can result in chemotaxis of neutrophils, increased proliferation and invasion of cancer cells, and precocious differentiation of T cells. Additionally, FasL has been shown to contribute to the pathogenesis of many human diseases including cancer, myocardial infarction, stroke, hepatic and kidney ischemia reperfusion injury, sepsis, interstitial lung disease, coronavirus disease 2019 (COVID-19), and autoimmune diseases. In patients with cancer, FasL contributes to disease progression through induction of apoptosis of tumor antigen-reactive infiltrating lymphocytes, precocious differentiation of T cells, and increased proliferation and invasiveness of tumor cells.

Despite the role for FasL in multiple human diseases and disorders, no clinically approved FasL-targeted therapeutics are currently available. Thus, a need exhibits for a potent inhibitor of FasL.

### SUMMARY

The present disclosure describes monoclonal antibodies that specifically bind to and block the function of Fas ligand (FasL). Also described herein is use of the disclosed antibodies in the development of therapeutics for the treatment of diseases and conditions associated with the Fas/FasL signaling pathway. The disclosed antibodies bind FasL with higher affinity and block FasL with greater potency than previously described drugs targeting FasL, and thus fulfill an unmet need.

Provided herein are monoclonal antibodies that specifically bind FasL. In some aspects, the FasL-specific monoclonal antibodies include the complementarity determining region (CDR) sequences of antibody M3T01, M3T02 or M3T03. In other aspects, the FasL-specific monoclonal antibody is an antibody that binds to the same epitope as M3T01, M3T02 or M3T03.

Also provided herein are conjugates that include a disclosed FasL-specific monoclonal antibody. In some aspects, provided are fusion proteins, multi-specific antibodies, chimeric antigen receptors (CARs), CAR-expressing immune cells, immunoconjugates, antibody-drug conjugates (ADCs), and antibody-nanoparticle conjugates that include a monoclonal antibody disclosed herein.

Further provided herein are nucleic acid molecules and vectors encoding the disclosed monoclonal antibodies, fusion proteins (such as Fc fusions), multi-specific antibodies (such as bi-specific antibodies), CARs, and immunoconjugates (such as immunotoxins).

Compositions and kits that include a pharmaceutically acceptable carrier and a FasL-specific monoclonal antibody, fusion protein, multi-specific antibody, CAR, immunoconjugate, ADC, antibody-nanoparticle conjugate, isolated nucleic acid molecule or vector disclosed herein are also provided by the present disclosure.

Also provided are methods of inhibiting Fas ligand in a subject in need thereof. In some aspects, the method includes administering to the subject a therapeutically effective amount of a monoclonal antibody, fusion protein, multi-specific antibody, CAR, CAR-expressing cell, immunoconjugate, ADC, antibody-nanoparticle conjugate, isolated nucleic acid molecule, vector or composition disclosed herein.

Further provided is a method of treating cancer, sepsis, myocardial infarction, stroke, hepatic ischemia-reperfusion injury, kidney ischemia-reperfusion injury, interstitial lung disease, autoimmune disease, or coronavirus disease 2019 (COVID-19) in a subject. In some aspects, the method includes administering to the subject a therapeutically effective amount of a monoclonal antibody, fusion protein, multi-specific antibody, CAR, CAR-expressing cell, immunoconjugate, ADC, antibody-nanoparticle conjugate, isolated nucleic acid molecule, vector or composition disclosed herein.

The foregoing and other objects and features of the disclosure will become more apparent from the following detailed description, which proceeds with reference to the accompanying figures.

### BRIEF DESCRIPTION OF THE DRAWINGS

FIGS. 1A-1B: Binding affinity of M3T01 to soluble human Fas ligand (FasL) measured using a BIACORE™ 8K. Sensorgrams of antibody to antigen (FIG. 1A) and affinity measurements (FIG. 1B) are shown.

FIG. 2A: Binding of M3T01 to soluble FasL measured by ELISA.

FIG. 2B: Binding of M3T01 to cell surface FasL across multiple species (human, cynomolgus macaque, rat, and mouse). M3T01 staining of HEK293T cells transfected with vectors expressing FasL from various species was measured by flow cytometry.

FIG. 3: M3T01 epitope on recombinant soluble FasL. The M3T01 epitope is a conformational epitope spanning R144 through Y189 of human FasL (numbered with reference to SEQ ID NO: 17). Residues of human, cynomolgus macaque, mouse, rat, rabbit, and pig FasL that are part of the conformational epitope are boxed. Shown are partial sequences of human FasL (residues 141-192 of SEQ ID NO: 17), cynomolgus macaque FasL (residues 140-191 of SEQ ID NO: 18), mouse FasL (residues 139-190 of SEQ ID NO: 19), rat FasL (residues 138-189 of SEQ ID NO: 20), rabbit FasL (residues 145-196 of SEQ ID NO: 21), and pig FasL (residues 142-193 of SEQ ID NO: 22).

FIG. 4: Inhibition of FasL-mediated apoptosis. HEK293T cells overexpressing human FasL (effector cells) were co-cultured with Jurkat T cells (target cells). M3T01 or soluble CD95-Fc was added to the cell culture at varying concentrations. Apoptosis of Jurkat cells was measured by annexin-V externalization by flow cytometry. Data is reported as the percentage of blocking apoptosis.

### SEQUENCE LISTING

The amino acid sequences listed in the accompanying sequence listing are shown using standard single letter code for amino acids, as defined in 37 C.F.R. 1.822. In the accompanying sequence listing:

SEQ ID NO: 1 is the amino acid sequence of the M3T01 VH domain.

SEQ ID NO: 2 is the amino acid sequence of the M3T01 VL domain.

SEQ ID NO: 3 is the amino acid sequence of the M3T02 VH domain.

SEQ ID NO: 4 is the amino acid sequence of the M3T02 VL domain.

SEQ ID NO: 5 is the amino acid sequence of the M3T03 VH domain.

SEQ ID NO: 6 is the amino acid sequence of the M3T03 VL domain.

SEQ ID NO: 7 is the amino acid sequence of a modified (S228P) human IgG4 constant region. SEQ ID NO: 8 is the amino acid sequence of the human kappa light chain constant region.

SEQ ID NO: 9 is the amino acid sequence of the M3T01 heavy chain.

SEQ ID NO: 10 is the amino acid sequence of the M3T01 light chain.

SEQ ID NO: 11 is the amino acid sequence of the M3T02 heavy chain.

SEQ ID NO: 12 is the amino acid sequence of the M3T02 light chain.

SEQ ID NOs: 13-15 are amino acid sequences of variant VH domains.

SEQ ID NO: 16 is the amino acid sequence of a variant VL domain.

SEQ ID NO: 17 is the amino acid sequence of human FasL.

MQQPFNYPYPQIYWVDSSASSPWAPPGTVLPCPTSVPRRPGQRRPPPPP
PPPPPPPPPPPPLPPLPLPPLKKRGNHSTGLCLLVMFFMVLVALVGLGL
GMFQLFHLQKELAELRESTSQMHTASSLEKQIGHPSPPPEKKELRKVAH
LTGKSNSRSMPLEWEDTYGIVLLSGVKYKKGGLVINETGLYFVYSKVYF
RGQSCNNLPLSHKVYMRNSKYPQDLVMMEGKMMSYCTTGQMWARSSYLG
AVFNLTSADHLYVNVSELSLVNFEESQTFFGLYKL

SEQ ID NO: 18 is the amino acid sequence of cynomolgus macaque FasL.

MQQPFNYPYPQIYWVDSSASSPWAPPGTVLPCPTSVPRRPGQRRPPPPP
PPPPLPPPPPSPLPPLPLPPLKKRGNHSTGLCLLVMFFMVLVALVGLGL
GMFQLFHLQKELAELRESTSQKHTASSLEKQIGHPSPPPEKKEQRKVAH
LTGKPNSRSMPLEWEDTYGIVLLSGVKYKKGGLVINETGLYFVYSKVYF
RGQSCTNLPLSHKVYMRNSKYPQDLVMMEGKMMSYCTTGQMWAHSSYLG
AVFNLTSADHLYVNVSELSLVNFEESQTFFGLYKL

SEQ ID NO: 19 is the amino acid sequence of mouse FasL.

MQQPMNYPCPQIFWVDSSATSSWAPPGSVFPCPSCGPRGPDQRRPPPPP
PPVSPLPPPSQPLPLPPLTPLKKKDHNTNLWLPVVFFMVLVALVGMGLG
MYQLFHLQKELAELREFTNQSLKVSSFEKQIANPSTPSEKKEPRSVAHL
TGNPHSRSIPLEWEDTYGTALISGVKYKKGGLVINETGLYFVYSKVYFR
GQSCNNQPLNHKVYMRNSKYPEDLVLMEEKRLNYCTTGQIWAHSSYLGA
VFNLTSADHLYVNISQLSLINFEESKTFFGLYKL

SEQ ID NO: 20 is the amino acid sequence of rat FasL.

MQQPVNYPCPQIYWVDSSATSPWAPPGSVFSCPSSGPRGPGQRRPPPPP
PPPSPLPPPSQPPPLPPLSPLKKKDNIELWLPVIFFMVLVALVGMGLGM
YQLFHLQKELAELREFTNHSLRVSSFEKQIANPSTPSETKKPRSVAHLT
GNPRSRSIPLEWEDTYGTALISGVKYKKGGLVINEAGLYFVYSKVYFRG
QSCNSQPLSHKVYMRNFKYPGDLVLMEEKKLNYCTTGQIWAHSSYLGAV
FNLTVADHLYVNISQLSLINFEESKTFFGLYKL

SEQ ID NO: 21 is the amino acid sequence of rabbit FasL.

MQQPFSYPYPQIYWVDSTASSPWAPPGSVLPCPSSVPERPGQRRPPPPL
PPPPPPLPPPPLPPLPPLPPLPPPPLKKRKDHSTGLCLLLMFFMVLVAX
VGLGWDVQLYHLQXELAELRESFSQRHTASSSMEKQTAHPSPPQEKKET
KKVAHLTGKSNSRSNPLEWEDTYGIALVSGLKYKKGNLVINDTGLYFVY
SKVYFRGQSCSNQPLTHKVYMKNSKYHQDLMLMEEKMMNYCTTGQMWAR
SSYLGAVFNLTSADHVYVNVSEISLVNFEESKTFFGLYKL

SEQ ID NO: 22 is the amino acid sequence of pig FasL.

MQQPFNYPYPQIFWVDSSATSPWASPGSVFPCPASVPGRPGQRRPPPPP
PPPPPPPTLLPSRPLPPLPPPSLKKKRDHNAGLCLLVMFFMVLVALVGL
GLGMFQLFHLQKELTELRESASQRHTESSLEKQIGHPNLPSEKKELRKV
AHLTGKPNSRSIPLEWEDTYGIALVSGVKYMKGSLVINDTGLYFVYSKV
YFRGQYCNNQPLSHKVYTRNSRYPQDLVLMEGKMMNYCTTGQMWARSSY
LGAVFNLTSADHLYVNVSELSLVNFEESKTFFGLYKL

### DETAILED DESCRIPTION

### I. Abbreviations

ADC antibody-drug conjugate
CAR chimeric antigen receptor
CDR complementarity determining region
ELISA enzyme-linked immunosorbent assay
FR framework
FasL Fas ligand
VH variable heavy
VL variable light
PBMC peripheral blood mononuclear cell
SPR surface plasmon resonance
TCR T cell receptor

### II. Terms and Methods

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various aspects, the following explanations of terms are provided:

Administration: To provide or give a subject an agent, such as a monoclonal antibody (e.g., a Fas ligand-specific monoclonal antibody) provided herein, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes.

Antibody: A polypeptide ligand comprising at least one variable region that recognizes and binds (such as specifically recognizes and specifically binds) an epitope of an antigen, such as Fas ligand. Mammalian immunoglobulin molecules are composed of a heavy (H) chain and a light (L) chain, each of which has a variable region, termed the variable heavy (VH) region and the variable light (VL) region, respectively. Together, the VH region and the VL region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles and is functionally similar to mammalian IgG and IgE. IgW and IgNAR antibodies are produced by cartilaginous fish, while IgX antibodies are found in amphibians.

Antibody variable regions contain framework regions (FR) and hypervariable (HV) regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8: e1002388, 2012; and Kunik et al., Nucleic Acids Res 40 (Web Server issue): W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, VH domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Shark V_{NAR} are comprised of the following regions (N-terminal to C-terminal): FR1-CDR1-FR2-HV2-FR3a-HV4-FR3b-CDR3-FR4. The positions of CDR1 and CDR3 of V_{NAR} antibodies can be determined, for example, using IMGT. HV2 and HV4 can be determined, for example, using annotation described in Stanfield et al. (Science 305:1770-1773, 2004) and Fennell et al. (J Mol Biol 400:155-170, 2010). Camelid VAH antibodies are produced by several species including camel, llama, alpaca, dromedary, and guanaco, which produce heavy chain antibodies that are naturally devoid of light chains. Camel V_{H}H are comprised of the following regions (N-terminal to C-terminal): FR1-CDR1-FR2-CDR2-FR3-CDR3-FR4. Camel V_{H}H CDR residues can be determined, for example, according to IMGT, Kabat or Paratome.

A “monoclonal antibody” is an antibody produced by a single clone of lymphocytes or by a cell into which the coding sequence of a single antibody has been transfected. The term “monoclonal antibody” refers to both full immunoglobulin molecules (e.g., IgG1, IgG4, IgA, IgM and the like), antigen-binding fragments of IgG molecules (e.g., scFv and Fab), and/or single-domain antibodies (such as VH single domain antibodies or camelid V_{H}H nanobodies).

A “chimeric antibody” has framework residues from one species, such as human, and CDRs (which generally confer antigen binding) from another species.

A “humanized” antibody is an immunoglobulin including a human framework region and one or more CDRs from a non-human (for example a camel, llama, mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Methods for measuring antibody competition are known, and include for example, ELISA and biolayer interferometry.

Antibody-drug conjugate (ADC): A molecule that includes an antibody (or antigen-binding fragment of an antibody) conjugated to a drug, such as a cytotoxic agent. ADCs can be used to specifically target a drug to particular cells through specific binding of the antibody to a target antigen expressed on the cell surface. Exemplary drugs for use with ADCs include anti-microtubule agents (such as maytansinoids, auristatin E and auristatin F) and interstrand crosslinking agents (for example, pyrrolobenzodiazepines; PBDs).

Autoimmune disease: A disorder in which the immune system produces an immune response (for example, a B cell or a T cell response) against an endogenous antigen, with consequent injury to tissues. Examples of autoimmune diseases included, but are not limited to, rheumatoid arthritis, Hashimoto's thyroiditis, pernicious anemia, Addison's disease, type I diabetes, systemic lupus erythematosus, dermatomyositis, Sjögren's syndrome, multiple sclerosis, myasthenia gravis, Reiter's syndrome, and Grave's disease.

Binding affinity: Affinity of an antibody for an antigen. In one aspect, affinity is calculated by a modification of the Scatchard method. In another aspect, binding affinity is measured by an antigen/antibody dissociation rate. In another aspect, a high binding affinity is measured by a competition radioimmunoassay. In another aspect, binding affinity is measured by ELISA. In some aspects, binding affinity is measured using the Octet system (Creative Biolabs), which is based on bio-layer interferometry (BLI) technology. In other aspects, Kd is measured using surface plasmon resonance (SPR) assays using a BIACORE™ 8K, a BIACORES-2000 or a BIACORES-3000 (BIAcore, Inc., Piscataway, N.J.). In other aspects, antibody affinity is measured by flow cytometry or by SPR. An antibody that “specifically binds” an antigen (such as Fas ligand) is an antibody that binds the antigen with high affinity and does not significantly bind other unrelated antigens. In some examples, a monoclonal antibody (such as an anti-Fas ligand antibody provided herein) specifically binds to its target with an equilibrium constant (Kd) of 10 nM or less, such as 9 nM or less, 8 nM or less, 7 nM or less, 6 nM or less, 5 nM or less, 4 nM or less, 3 nM or less, 2.5 nM or less, 2.4 nM or less, 2.3 nM or less, 2.1 nM or less, 2 nM or less, 1.9 nM or less, 1.8 nM or less, 1.7 nM or less, 1.6 nM or less, 1.5 nM or less, 1.4 nM or less, 1.3 nM or less, 1.2 nM or less, 1.1 nM or less, 1 nM or less, 975 pM or less, 950 pM or less, 925 pM or less, or 900 nM or less.

Bispecific antibody: A recombinant protein that includes antigen-binding fragments of two different monoclonal antibodies and is thereby capable of binding two different antigens or two different epitopes of the same antigen (such as Fas ligand). Similarly, a multi-specific antibody is a recombinant protein that includes antigen-binding fragments of at least two different monoclonal antibodies, such as two, three or four different monoclonal antibodies.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic cancer” refers to cancer cells that have left the original tumor site (e.g., a lung) and migrate to other parts of the body (e.g., a lung cancer cell migrating to the liver, brain, or bone) for example via the bloodstream or lymph system.

Exemplary cancers include, but are not limited to, solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi's sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck cancers, and lymphatic tumors (including B-cell and T-cell malignant lymphoma).

Exemplary tumors of the blood include, for example, lymphatic tumors, white blood cell tumors, and other types of leukemia. In particular examples, the tumor is a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), or a lymphoma (such as Hodgkin's lymphoma and non-Hodgkin's lymphoma), and myelomas).

Chimeric antigen receptor (CAR): A chimeric molecule that includes an antigen-binding portion (such as a monoclonal antibody or antigen-binding fragment thereof) and a signaling domain, such as a signaling domain from a T cell receptor (for example, CD33). Typically, CARs are comprised of an antigen-binding moiety, a transmembrane domain and an endodomain. The endodomain typically includes a signaling chain having an immunoreceptor tyrosine-based activation motif (ITAM), such as CD3 or FcεRIγ. In some instances, the endodomain further includes the intracellular portion of at least one additional co-stimulatory domain, such as CD28, 4-1BB (CD137), ICOS, OX40 (CD134), CD27, MYD88-CD40, KIR2DS2 and/or DAP10.

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. Camel (V_{H}H) single-domain antibodies, VH single-domain antibodies and V_{L} single-domain antibodies contain three CDRs, referred to as CDR1, CDR2 and CDR3.

Conservative variant: A protein containing conservative amino acid substitutions that do not substantially affect or decrease the affinity of a protein, such as an antibody to Fas ligand. For example, a monoclonal antibody that specifically binds to Fas ligand can include at most about 1, at most about 2, at most about 5, and most about 10, or at most about 15 conservative substitutions and specifically bind Fas ligand. The term “conservative variant” also includes the use of a substituted amino acid in place of an unsubstituted parent amino acid, provided that antibody specifically binds Fas ligand. Non-conservative substitutions are those that may reduce an activity or binding to Fas ligand.

Conservative amino acid substitution tables providing functionally similar amino acids are well known. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association; includes both in solid and liquid form.

Coronavirus disease 2019 (COVID-19): The disease caused by the human betacoronavirus SARS-CoV-2. Symptoms of COVID-19 include, for example, fever, chills, dry cough, shortness of breath, fatigue, muscle/body aches, headache, new loss of taste or smell, sore throat, nausea or vomiting, and diarrhea. Patients with severe COVID-19 can develop pneumonia, multi-organ failure, and death.

Cytotoxic agent: Any drug or compound that kills cells.

Cytotoxicity: The toxicity of a molecule, such as an immunotoxin, to the cells intended to be targeted, as opposed to the cells of the rest of an organism. In contrast, the term “toxicity” refers to toxicity of an immunotoxin to cells other than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Drug: Any compound used to treat, ameliorate or prevent a disease or condition in a subject. In some aspects herein, the drug is an anti-cancer agent.

Effector molecule: The portion of a chimeric molecule that is intended to have a desired effect on a cell to which the chimeric molecule is targeted. Effector molecule is also known as an effector moiety (EM), therapeutic agent, diagnostic agent, or similar terms. Therapeutic agents (or drugs) include such compounds as nucleic acids, proteins, peptides, amino acids or derivatives, glycoproteins, radioisotopes, photon absorbers, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to a targeting moiety, such as an anti-Fas ligand antibody, may be an encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are known. Diagnostic agents or moieties include radioisotopes and other detectable labels. Detectable labels useful for such purposes include radioactive isotopes such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I, fluorophores, chemiluminescent agents, and enzymes.

Fas ligand (FasL): A transmembrane protein that is a member of the tumor necrosis factor superfamily. Fas ligand, which is primarily expressed by activated T cells and natural killer cells, induces apoptosis upon binding to Fas, which is ubiquitously expressed throughout the body, but is particularly abundant in the thymus, liver, heart and kidney (Peter et al., Cell Death Differ 22 (4): 549-559, 2015). The Fas/FasL signaling pathway plays an important role in immune system regulation, including activation-induced cell death of T cells and cytotoxic T lymphocyte (CTL)-induced cell death. However, these proteins are also involved in tumor-promoting activities. In particular, they have been found to be critical survival factors for cancer cells and are capable of protecting and promoting cancer stem cells (Peter et al., Cell Death Differ 22 (4): 549-559, 2015). Fas ligand is also known as CD95 ligand (CD95L) and tumor necrosis factor ligand superfamily member 6 (TNFSF6). Nucleotide and amino acid sequences for FasL are publicly available, such as under NCBI Gene ID 356. An exemplary human FasL amino acid sequence is set forth herein as SEQ ID NO: 17.

Framework region: Amino acid sequences interposed between CDRs (and/or hypervariable regions).

Fusion protein: A protein comprising at least a portion of two different (heterologous) proteins.

Heterologous: Originating from a separate genetic source or species.

Host cell: Cells in which a vector can be propagated and its DNA expressed. The cell may be prokaryotic or eukaryotic. In some examples, the prokaryotic cell is an E. coli cell. In some examples, the eukaryotic cell is a human cell, such as a human embryonic kidney (HEK) cell or HEK293 T cell. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4+ response or a CD8+ response. In another aspect, the response is a B cell response, and results in the production of specific antibodies.

Immunoconjugate: A covalent linkage of an effector molecule to an antibody (such as a monoclonal antibody specific for FasL) or functional fragment thereof. The effector molecule can be, for example, a detectable label, a photon absorber (such as IR700), or a toxin (to form an immunotoxin, such as an immunotoxin comprising Pseudomonas exotoxin or a variant thereof). Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain Ia of PE and the B chain of DT) and replacing it with a different targeting moiety, such as an antibody. In one aspect, an antibody is joined to an effector molecule. In another aspect, an antibody joined to an effector molecule is further joined to a lipid or other molecule, such as to increase its half-life in the body. The linkage can be either by chemical or recombinant means. In one aspect, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. The term “conjugated” or “linked” refers to making two polypeptides into one contiguous polypeptide molecule.

Immunoliposome: A liposome with antigen-binding monoclonal antibodies (such as antibodies specific for FasL) conjugated to its surface. Immunoliposomes can carry cytotoxic agents or other drugs to antibody-targeted cells, such as tumor cells.

Interstitial lung disease: A group of chronic lung disorders characterized by inflammation and scarring that prevent the lung tissue from receiving sufficient oxygen.

Ischemia: A vascular phenomenon in which a decrease in the blood supply to a bodily organ, tissue, or part is caused, for instance, by constriction or obstruction of one or more blood vessels. Ischemia sometimes results from vasoconstriction, thrombosis, or embolism. Ischemia can lead to direct ischemic injury, e.g., tissue damage due to cell death caused by reduced oxygen supply.

Ischemia-reperfusion injury: Tissue injury that occurs after blood flow is restored to an ischemic site. The absence of oxygen and nutrients from the blood during an ischemic event leads to a condition in which the restoration of circulation produces inflammation and oxidative damage through the induction of oxidative stress.

Isolated: An “isolated” biological component, such as a nucleic acid, protein (including antibodies) or organelle, has been substantially separated or purified away from other biological components in the environment (such as a cell) in which the component occurs, for example other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids. In some examples, an isolated biological component is at least 90% pure, at least 95%, at least 98%, at least 99%, at least 99.9%, at least 99.99% or 100% pure.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide or other molecule to a polypeptide, such as an antibody. The linkage can be either by chemical or recombinant means. “Chemical means” refers to a reaction between the antibody moiety and the effector molecule such that there is a covalent bond formed between the two molecules to form one molecule.

Myocardial infarction: A medical condition that occurs when one or more areas of the heart do not receive sufficient oxygen. Also known as a “heart attack.”

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are known to one of skill in the art. Remington: The Science and Practice of Pharmacy, 22^{nd} ed., London, UK: Pharmaceutical Press, 2013, describes compositions and formulations suitable for pharmaceutical delivery of the polypeptides, antibodies and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease, such as cancer.

Sepsis: An extreme immune response to an infection or injury. Most cases of sepsis are caused by bacterial infections, but sepsis can also be caused by viral infections (such as SARS-CoV-2 and influenza virus), fungal infections or traumatic injury. If not timely treated, sepsis can lead to tissue damage, organ failure, septic shock and death.

Stroke: A medical condition that occurs when the blood supply to the brain (or a part of the brain) is interrupted or reduced, preventing brain tissue from receiving sufficient oxygen and nutrients.

Subject: Living multi-cellular vertebrate organisms, a category that includes both human and non-human animals (such as veterinary subjects or wild animals), for example birds, pigs, mice, rats, rabbits, sheep, horses, cows, dogs, cats, ferrets, deer, otters, bank voles, racoon dogs, tree shrews, fruit bats, hamsters, mink, and non-human primates (e.g., rhesus macaques, cynomolgus macaques, baboons, grivets, and common marmosets). In some examples, a subject has cancer, sepsis, or another condition suitable for treatment with an inhibitor of Fas ligand signaling. In one example, a subject has glioblastoma multiforme.

Synthetic: Produced by artificial means in a laboratory, for example a synthetic nucleic acid or protein (for example, an antibody) can be chemically synthesized in a laboratory.

Therapeutically effective amount: The amount of agent, such as a monoclonal antibody, that is alone (or in combination with other therapeutic agents) sufficient to prevent, treat (including prophylaxis), reduce and/or ameliorate the symptoms and/or underlying causes of a disease or disorder, for example to prevent, inhibit, and/or treat a cancer, sepsis, myocardial infarction, stroke, hepatic ischemia-reperfusion injury, kidney ischemia-reperfusion injury, interstitial lung disease, autoimmune disease, or COVID-19. In some aspects, a therapeutically effective amount is sufficient to reduce or eliminate a symptom of a disease, such as reduce tumor volume or tumor metastases.

For instance, this can be the amount necessary to inhibit or suppress growth of a tumor. In one aspect, a therapeutically effective amount is the amount necessary to eliminate, reduce the size, or prevent metastasis of a cancer, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment (or for example as compared to another treatment. When administered to a subject, a dosage will generally be used that will achieve target tissue concentrations (for example, in tumors) that has been shown to achieve a desired in vitro effect.

A therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements. In some aspects, the vector is a virus vector, such as a lentivirus vector, an adenovirus vector, or an adeno-associated virus (AAV).

### III. Monoclonal Antibodies Specific for Fas Ligand

Disclosed herein are monoclonal antibodies that specifically bind to and block the function of Fas ligand (FasL or CD95L). FasL binding to the Fas receptor (Fas or CD95) can induce either apoptotic or non-apoptotic signaling in the cell expressing the Fas receptor. Apoptotic signaling results in cell death of the cell expressing the Fas receptor. Non-apoptotic signaling through FasL/Fas receptor interactions can result in chemotaxis of neutrophils, increased proliferation and invasion of cancer cells, and precocious differentiation of T cells.

FasL contributes to the pathogenesis of many human diseases including, but not limited to, cancer, myocardial infarction, stroke, hepatic and kidney ischemia reperfusion injury, sepsis, interstitial lung disease, COVID-19, and autoimmune diseases. In cancer, FasL contributes to disease progression through induction of apoptosis of tumor antigen-reactive infiltrating lymphocytes, precocious differentiation of T cells, and increased proliferation and invasiveness of tumor cells. Thus, use of the disclosed antibodies for the treatment of diseases and disorders associated with FasL is described.

There are currently no approved drugs that target FasL for the treatment of FasL-associated diseases. Although Asunercept (APG101; Apogenix, Heidelberg, Germany), a fusion protein that includes the human Fas receptor and human IgG Fc, has been tested in clinical trials, this drug has a short half-life that requires weekly administration. In addition, the monoclonal antibodies disclosed herein bind FasL with much higher binding affinity and block FasL function with far greater potency than a soluble CD95-Fc fusion protein having the amino acid sequence of Asunercept.

The amino acid sequences of the VH and VL domains of M3T01, M3T02 and M3T03 are provided below and are set forth herein as SEQ ID NOs: 1-6. The location of each CDR, as determined by Kabat, is also shown below. Other numbering schemes, such as IMGT or Chothia, can also be used to determine the boundaries of each CDR. Also shown below are the amino acid sequence of a human IgG4 heavy chain constant region with an S228P substitution (SEQ ID NO: 7) to prevent Fab arm exchange, and the amino acid sequence of a human kappa light chain constant region (SEQ ID NO: 8). Amino acid sequences for the M3T01 and M3T02 heavy chain and light chain are also provided below (and set forth herein as SEQ ID NOs: 9-12)

M3T01 VH domain
(SEQ ID NO: 1)
QVQLVESGGGVVQPGRSLRLSCAASGFTFSTYGIHWVRQAPGKGLEWVA
VIWYDGSDKFYADSVKGRFTISRDNSKNTLYLQMNSLRAEDTAVYYCAR
DRDNWNYFDYWGQGTLVTVSS
M3T01 VL domain
(SEQ ID NO: 2)
DIQMTQSPSTLSASVGDRVTITCRASQSISSWLAWYQQKPGKAPKFLIY
QASSLESGVPSRFSGSGSGTEFTLTISSLQPDDFATYYCQQYNSYITFG
QGTRLEIK
M3T02 VH domain
(SEQ ID NO: 3)
QVQLVDSGGGVVQPGRSLRLSCAASGFTFSSYGMHWVRQAPGKGPEWVA
VIWYDGSNKYYADSVKGRFTISRDNSKNTLYLQMNSLRAEDTAVYYCAR
DRDNWNHFDYWGQGTLVTVSS
M3T02 VL domain
(SEQ ID NO: 4)
EIVMTQSPATLSVSPGERATLSCRASQSESSNLAWYQQKPGQAPRLLIY
GASTRATGIPARFSGSGSGTEFTLTISSLQSEDFAVYYCQQYNNWLTFG
GGTKVEIK
M3T03 VH domain
(SEQ ID NO: 5)
QIQLVQSGPDLKKPGETVKISCKASGYTFTNYGMNWVKKAPGKGLKWMG
WINTNTGEPSYAEEFKGRFAFSLETSAGTAYLHINNLKNEDTATYFCVK
YSRYYAMDFWGQGTSVTVSS
M3T03 VL domain
(SEQ ID NO: 6)
QAVVTQESALTTSPGETVTLTCRSSTGAVTTSNYANWVQEKPDHLFTGL
IGDTNNRAPGVPARFSGSLIGDKAALTITGAQTEDEAMYFCALWYSNHW
VFGGGTKLTVL

TABLE 1
Locations of the heavy chain and light chain CDRs

| Antibody Domain | SEQ ID NO: | CDR1 | CDR2 | CDR3 |
|---|---|---|---|---|
| M3T01 VH domain | 1 | 31-35 | 50-66 | 99-108 |
| M3T01 VL domain | 2 | 24-34 | 50-56 | 89-96 |
| M3T02 VH domain | 3 | 31-35 | 50-66 | 99-108 |
| M3T02 VL domain | 4 | 24-34 | 50-56 | 89-96 |
| M3T03 VH domain | 5 | 31-35 | 50-66 | 99-107 |
| M3T03 VL domain | 6 | 23-36 | 52-58 | 91-99 |

Human IgG4 heavy chain constant region
(SEQ ID NO: 7)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
Human kappa light chain constant region
(SEQ ID NO: 8)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC
M3T01 heavy chain
(SEQ ID NO: 9)
QVQLVESGGGVVQPGRSLRLSCAASGFTFSTYGIHWVRQAPGKGLEWVA
VIWYDGSDKFYADSVKGRFTISRDNSKNTLYLQMNSLRAEDTAVYYCAR
DRDNWNYFDYWGQGTLVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSLGK
VH domain: residues 1-119
IgG4 constant region: residues 120-446
M3T01 light chain
(SEQ ID NO: 10)
DIQMTQSPSTLSASVGDRVTITCRASQSISSWLAWYQQKPGKAPKFLIY
QASSLESGVPSRFSGSGSGTEFTLTISSLQPDDFATYYCQQYNSYITFG
QGTRLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
VL domain: residues 1-106
Kappa constant region: residues 107-213
M3T02 heavy chain
(SEQ ID NO: 11)
QVQLVDSGGGVVQPGRSLRLSCAASGFTFSSYGMHWVRQAPGKGPEWVA
VIWYDGSNKYYADSVKGRFTISRDNSKNTLYLQMNSLRAEDTAVYYCAR
DRDNWNHFDYWGQGTLVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSLGK
VH domain: residues 1-119
IgG4 constant region: residues 120-446
M3T02 light chain
(SEQ ID NO: 12)
EIVMTQSPATLSVSPGERATLSCRASQSFSSNLAWYQQKPGQAPRLLIY
GASTRATGIPARFSGSGSGTEFTLTISSLOSEDFAVYYCOQYNNWLTFG
GGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
VL domain: residues 1-106
Kappa constant region: residues 107-213

Provided herein are monoclonal antibodies that specifically bind FasL. In some aspects, the FasL-specific antibody has a binding affinity for FasL of 10 nM or less, 9 nM or less, 8 nM or less, 7 nM or less 6 nM or less, 5 nM or less, 4 nM or less, 3 nM or less, 2 nM or less, 1.9 nM or less, 1.8 nM or less, 1.7 nM or less, 1.6 nM or less, 1.5 nM or less, 1.4 nM or less, 1.3 nM or less, 1.2 nM or less, 1.1 nM or less, 1 nM or less, 975 pM or less, 950 pM or less, 925 pM or less, or 900 nM or less. In some aspects, the monoclonal antibody includes a variable heavy (VH) domain and a variable light (VL) domain. In some examples, the monoclonal antibody includes at least a portion of the amino acid sequence set forth herein as SEQ ID NO: 1 and/or SEQ ID NO: 2, such as one or more (such as all three) CDR sequences from SEQ ID NO: 1 and/or one or more (such as all three) CDR sequences from SEQ ID NO: 2, as determined by any numbering scheme, such as IMGT, Kabat or Chothia, or any combination thereof. In other examples, the monoclonal antibody includes at least a portion of the amino acid sequence set forth herein as SEQ ID NO: 3 and/or SEQ ID NO: 4, such as one or more (such as all three) CDR sequences from SEQ ID NO: 3 and/or one or more (such as all three) CDR sequences from SEQ ID NO: 4, as determined by any numbering scheme, such as IMGT, Kabat or Chothia, or any combination thereof. In other examples, the monoclonal antibody includes at least a portion of the amino acid sequence set forth herein as SEQ ID NO: 5 and/or SEQ ID NO: 6, such as one or more (such as all three) CDR sequences from SEQ ID NO: 5 and/or one or more (such as all three) CDR sequences from SEQ ID NO: 6, as determined by any numbering scheme, such as IMGT, Kabat or Chothia, or any combination thereof.

In some aspects, the VH domain of the monoclonal antibody includes the CDR1, CDR2 and CDR3 sequences of SEQ ID NO: 1 and/or the VL domain of the monoclonal antibody includes the CDR1, CDR2 and CDR3 sequences of SEQ ID NO: 2. In other aspects, the VH domain of the monoclonal antibody includes the CDR1, CDR2 and CDR3 sequences of SEQ ID NO: 3 and/or the VL domain of the monoclonal antibody includes the CDR1, CDR2 and CDR3 sequences of SEQ ID NO: 4. In other aspects, the VH domain of the monoclonal antibody includes the CDR1, CDR2 and CDR3 sequences of SEQ ID NO: 5 and/or the VL domain of the monoclonal antibody includes the CDR1, CDR2 and CDR3 sequences of SEQ ID NO: 6. In some examples, the CDR sequences are determined using the Kabat, IMGT or Chothia numbering scheme, or a combination thereof. In particular examples, the CDR sequences are determined using Kabat.

In some aspects, the CDR1, CDR2 and CDR3 sequences of the VH domain of the monoclonal antibody respectively include residues 31-35, 50-66 and 99-108 of SEQ ID NO: 1 and/or the CDR1, CDR2 and CDR3 sequences of the VL domain of the monoclonal antibody respectively include residues 24-34, 50-56 and 89-96 of SEQ ID NO: 2. In some examples, the amino acid sequence of the VH domain is at least 90% identical (such as at least 91%, 92%, 93%, 94%, 95%, 96%, 97%, 98%, 99%, or 100% identical) to SEQ ID NO: 1 and includes residues 31-35, 50-66 and 99-108 of SEQ ID NO: 1 and/or the amino acid sequence of the VL domain is at least 90% identical (such as at least 91%, 92%, 93%, 94%, 95%, 96%, 97%, 98%, 99%, or 100% identical) to SEQ ID NO: 2 and includes residues 24-34, 50-56 and 89-96 of SEQ ID NO: 2. In particular examples, the amino acid sequence of the VH domain comprises or consists of SEQ ID NO: 1 and/or the amino acid sequence of the VL domain comprises or consists of SEQ ID NO: 2.

In other examples, the amino acid sequence of the VH domain comprises or consists of SEQ ID NO: 1, SEQ ID NO: 13, SEQ ID NO: 16, SEQ ID NO: 17 or SEQ ID NO: 18, and/or the amino acid sequence of the VL domain comprises or consists of SEQ ID NO: 2, SEQ ID NO: 14 or SEQ ID NO: 15. In particular examples, the amino acid sequence of the VH domain comprises or consists of SEQ ID NO: 13 and the amino acid sequence of the VL domain comprises or consists of SEQ ID NO: 14. In other particular examples, the amino acid sequence of the VH domain comprises or consists of SEQ ID NO: 1 and the amino acid sequence of the VL domain comprises or consists of SEQ ID NO: 15. In other particular examples, the amino acid sequence of the VH domain comprises or consists of SEQ ID NO: 16 and the amino acid sequence of the VL domain comprises or consists of SEQ ID NO: 2. In other particular examples, the amino acid sequence of the VH domain comprises or consists of SEQ ID NO: 17 and the amino acid sequence of the VL domain comprises or consists of SEQ ID NO: 2. In other particular examples, the amino acid sequence of the VH domain comprises or consists of SEQ ID NO: 18 and the amino acid sequence of the VL domain comprises or consists of SEQ ID NO: 2.

In other aspects, the CDR1, CDR2 and CDR3 sequences of the VH domain of the monoclonal antibody respectively include residues 31-35, 50-66 and 99-108 of SEQ ID NO: 3 and/or the CDR1, CDR2 and CDR3 sequences of the VL domain of the monoclonal antibody respectively include residues 24-34, 50-56 and 89-96 of SEQ ID NO: 4. In some examples, the amino acid sequence of the VH domain is at least 90% identical (such as at least 91%, 92%, 93%, 94%, 95%, 96%, 97%, 98%, 99%, or 100% identical) to SEQ ID NO: 3 and includes residues 31-35, 50-66 and 99-108 of SEQ ID NO: 3 and/or the amino acid sequence of the VL domain is at least 90% identical (such as at least 91%, 92%, 93%, 94%, 95%, 96%, 97%, 98%, 99%, or 100% identical) to SEQ ID NO: 4 and comprises residues 24-34, 50-56 and 89-96 of SEQ ID NO: 4. In particular examples, the amino acid sequence of the VH domain comprises or consists of SEQ ID NO: 3 and/or the amino acid sequence of the VL domain comprises or consists of SEQ ID NO: 4.

In other aspects, the CDR1, CDR2 and CDR3 sequences of the VH domain of the monoclonal antibody respectively include residues 31-35, 50-66 and 99-107 of SEQ ID NO: 5 of SEQ ID NO: 5; and/or the CDR1, CDR2 and CDR3 sequences of the VL domain of the monoclonal antibody respectively include residues 23-36, 52-58 and 91-99 of SEQ ID NO: 6. In some examples, the amino acid sequence of the VH domain is at least 90% identical (such as at least 91%, 92%, 93%, 94%, 95%, 96%, 97%, 98%, 99%, or 100% identical) to SEQ ID NO: 5 and includes residues 31-35, 50-66 and 99-107 of SEQ ID NO: 5 and/or the amino acid sequence of the VL domain is at least 90% identical (such as at least 91%, 92%, 93%, 94%, 95%, 96%, 97%, 98%, 99%, or 100% identical) to SEQ ID NO: 6 and includes residues 23-36, 52-58 and 91-99 of SEQ ID NO: 6. In particular examples, the amino acid sequence of the VH domain comprises or consists of SEQ ID NO: 5 and/or the amino acid sequence of the VL domain comprises or consists of SEQ ID NO: 6.

In some aspects, the monoclonal antibody is an antigen-binding fragment selected from an Fab fragment, an Fab′ fragment, an F (ab)′2 fragment, a single chain variable fragment (scFv) and a disulfide stabilized variable fragment (dsFv). In other aspects, the monoclonal antibody is an IgG, such as an IgG4 or IgG1.

In some aspects, the monoclonal antibody includes or further includes a heavy chain constant region and/or a light chain constant region. In some examples, the heavy chain constant region is a human IgG4 heavy chain constant region. In some examples, the heavy chain constant region has one or more modifications relative to a wild-type heavy chain to increase the half-life, stability and/or function of the monoclonal antibody, such as the S228P substitution in the human IgG4 heavy chain to prevent Fab arm exchange. In particular examples, the amino acid sequence of the heavy chain constant region is at least 85%, at least 90%, at least 95%, at least 96%, at least 97%, at least 98% or at least 99% identical to SEQ ID NO: 7. In specific non-limiting examples, the amino acid sequence of the heavy chain constant region comprises or consists of SEQ ID NO: 7.

In some examples, the light chain constant region is a human kappa light chain constant region. In some examples, the light chain constant region has one or more modifications relative to a wild-type light chain constant region to increase the half-life, stability and/or function of the monoclonal antibody. In particular examples, the amino acid sequence of the light chain constant region is at least 85%, at least 90%, at least 95%, at least 96%, at least 97%, at least 98% or at least 99% identical to SEQ ID NO: 8. In some examples, the amino acid sequence of the light chain constant region comprises or consists of SEQ ID NO: 8.

In some aspects, the monoclonal antibody includes a heavy chain and a light chain, and the amino acid sequence of the heavy chain comprises or consists of SEQ ID NO: 9 and/or the amino acid sequence of the light chain comprises or consists of SEQ ID NO: 10.

In other aspects; the monoclonal antibody includes a heavy chain and a light chain, and the amino acid sequence of the heavy chain comprises or consists of SEQ ID NO: 11 and/or the amino acid sequence of the light chain comprises or consists of SEQ ID NO: 12.

In some aspects, the monoclonal antibody is a human antibody. In other aspects, the monoclonal antibody is a humanized antibody. In yet other aspects, the monoclonal antibody is a chimeric antibody.

Also provided herein are monoclonal antibodies that bind to the same epitope on Fas ligand to which the M3T01, M3T02 or M3T03 antibody binds (see also section IV). In some aspects, the monoclonal antibody binds to the same epitope as a Fas ligand-specific monoclonal antibody that includes a heavy chain comprising the amino acid sequence of SEQ ID NO: 9 and a light chain comprising the amino acid sequence of SEQ ID NO: 10. In some examples, the epitope is a conformational epitope spanning R144 to Y189 of Fas ligand set forth as SEQ ID NO: 17.

Further provided herein are fusion proteins that include a disclosed Fas ligand-specific monoclonal antibody and a heterologous protein. In some examples, the heterologous protein is an Fc protein, such as human Fc. In other examples, the heterologous protein is a protein tag, such as a myc tag, His tag, HA tag, or FLAG tag. In other examples, the heterologous protein is an affinity tag, such as chitin binding protein, maltose binding protein, or glutathione-S-transferase (GST).

Further provided are multi-specific antibodies that include a Fas ligand-specific monoclonal antibody disclosed herein and at least one additional monoclonal antibody. In some aspects, the multi-specific monoclonal antibody is a bispecific or trispecific monoclonal antibody. The at least one additional monoclonal antibody can bind a different epitope on Fas ligand or can bind to a different antigen. Multi-specific antibodies are further described in section V.

Also provided herein are chimeric antigen receptors (CARs) that include a monoclonal antibody disclosed herein. In some aspects, the CAR further includes a hinge region, a transmembrane domain, a costimulatory signaling moiety, a signaling domain, or any combination thereof. Further provided are cells, such as immune cells, expressing a FasL-specific CAR. In some examples, the immune cell is a T lymphocyte, such as a CTL, a B cell, a natural killer (NK) cell, or a macrophage. In some examples, the cells are allogeneic cells, such as allogeneic cells obtained from a healthy donor. CARs and CAR-expressing cells are further described in section VI.

Further provided are immunoconjugates that include a FasL-specific monoclonal antibody disclosed herein and an effector molecule. In some aspects, the effector molecule is a toxin, a detectable label, or a photon absorber. Immunoconjugates are further described in section VII.

Also provided are antibody-drug conjugates (ADCs) that include a drug conjugated to a FasL-specific monoclonal antibody disclosed herein. In some aspects, the drug is a small molecule, for example an anti-microtubule agent, an anti-mitotic agent and/or a cytotoxic agent. ADCs are further described in section VIII.

Further provided are antibody-nanoparticle conjugates that include a nanoparticle conjugated to a FasL-specific monoclonal antibody disclosed herein. In some aspects, the nanoparticle comprises a polymeric nanoparticle, nanosphere, nanocapsule, liposome, dendrimer, polymeric micelle, or niosome. Antibody-nanoparticle conjugates are further described in section IX.

Also provided herein are nucleic acid molecules that encode a monoclonal antibody, fusion protein, conjugate or multi-specific antibody disclosed herein. In some aspects, the nucleic acid molecule is operably linked to a promoter. Further provided are vectors that include a nucleic acid molecule disclosed herein, and cells that include a nucleic acid molecule or vector disclosed herein. The host cells can be, for example, mammalian cells, bacterial cells, or insect cells. Nucleic acid molecules and vectors are further described in section X.

Further provided are compositions that include a pharmaceutically acceptable carrier a monoclonal antibody, fusion protein, conjugate, multi-specific antibody, nucleic acid molecule, or vector disclosed herein. Compositions are further described in section XI.

Also provided herein are methods of inhibiting Fas ligand in a subject in need thereof. In some aspects, the method includes administering to the subject a therapeutically effective amount of a monoclonal antibody, fusion protein, conjugate, multi-specific antibody, nucleic acid molecule, vector, or composition disclosed herein. In some examples, the subject has cancer, sepsis, myocardial infarction, stroke, hepatic ischemia-reperfusion injury, kidney ischemia-reperfusion injury, interstitial lung disease, autoimmune disease, or COVID-19. In particular examples, the cancer is glioblastoma multiforme or a myelodysplastic syndrome.

Further provided are methods of treating cancer, sepsis, myocardial infarction, stroke, hepatic ischemia-reperfusion injury, kidney ischemia-reperfusion injury, interstitial lung disease, autoimmune disease, or COVID-19 in a subject. In some aspects, the method includes administering to the subject a therapeutically effective amount of a monoclonal antibody, fusion protein, conjugate, multi-specific antibody, nucleic acid molecule, vector, or composition disclosed herein. In some examples, the cancer is glioblastoma multiforme or a myelodysplastic syndrome. Methods are further described in section XII.

### IV. Identification of Antibodies that Bind the Same Epitope on Fas Ligand

Also provided herein are monoclonal antibodies that bind to the same epitope on Fas ligand to which the M3T01, M3T02 or M3T03 antibody binds. Antibodies that bind to such an epitope can be identified based on their ability to cross-compete (for example, to competitively inhibit the binding of, in a statistically significant manner) with the M3T01, M3T02 or M3T03 antibodies provided herein in Fas ligand binding assays (such as those described in the Examples). An antibody “competes” for binding when the competing antibody inhibits Fas ligand binding of the M3T01, M3T02 or M3T03 antibody by more than 50%, in the presence of competing antibody concentrations higher than 10⁶×K_{D} of the competing antibody. In a certain aspect, the antibody that binds to the same epitope on Fas ligand as the M3T01, M3T02 or M3T03 antibody is a human monoclonal antibody. Human antibodies that bind to the same epitope on Fas ligand to which the M3T01, M3T02 or M3T03 antibody binds can be produced using various known techniques. Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For a review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies that bind to the same epitope on Fas ligand to which the M3T01, M3T02 or M3T03 antibody binds can also be made by hybridoma-based methods. Human myeloma and mouse-human heteromyeloma cell lines for the production of human monoclonal antibodies have been described (see, e.g., Kozbor J. Immunol., 133:3001, 1984; and Boerner et al., J. Immunol. 147:86, 1991). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005). Human antibodies may also be generated by isolating Fv clone variable domain sequences selected from human-derived phage display libraries. Such variable domain sequences may then be combined with a desired human constant domain.

Monoclonal antibodies that specifically bind to the same epitope on Fas ligand as M3T01, M3T02 or M3T03 can also be isolated by screening combinatorial libraries for antibodies with the desired binding characteristics. For example, a variety of methods are known for generating phage display libraries and screening such libraries for antibodies possessing the desired binding characteristics. Such methods are reviewed in, e.g., Hoogenboom, Methods Mol Biol 178:1-37, 2002.

### V. Multi-Specific Antibodies

Multi-specific antibodies are recombinant proteins comprised of two or more monoclonal antibodies (or antigen-binding fragments thereof) of two or more different monoclonal antibodies. For example, bispecific antibodies can be comprised of antigen-binding fragments of two different monoclonal antibodies. Thus, bispecific antibodies bind two different antigens (or two different epitopes of the same antigen) and trispecific antibodies bind three different antigens or epitopes.

Provided herein are multi-specific, such as trispecific or bispecific, monoclonal antibodies comprising a FasL-specific monoclonal antibody. In some aspects, the multi-specific monoclonal antibody further includes a monoclonal antibody that specifically binds another protein. In other aspects, the multi-specific monoclonal antibody further includes a second monoclonal antibody that specifically binds a different epitope on FasL. Also provided are isolated nucleic acid molecules and vectors encoding the multi-specific antibodies, and host cells comprising the nucleic acid molecules or vectors. Thus, provided herein are methods for the treatment of cancer, sepsis, myocardial infarction, stroke, hepatic ischemia-reperfusion injury, kidney ischemia-reperfusion injury, interstitial lung disease, autoimmune disease, or COVID-19 (or any other disease or disorder enhanced by Fas/FasL signaling) in a subject by administering to the subject a therapeutically effective amount of the FasL-targeting multi-specific (such as bispecific) antibody.

### VI. Chimeric Antigen Receptors (CARs)

The disclosed monoclonal antibodies can be used to produce CARs and/or immune cells (such as T cells, B cells, natural killer (NK) cells, or macrophages) engineered to express CARs. In some aspects, the CARs include a binding moiety, an extracellular hinge and spacer element, a transmembrane region and an endodomain that performs signaling functions (Cartellieri et al., J Biomed Biotechnol 2010:956304, 2010; Dai et al., J Natl Cancer Inst 108 (7): djv439, 2016). In some instances, the binding moiety is an antigen binding fragment of a monoclonal antibody, such as a scFv. The spacer/hinge region typically includes sequences from IgG subclasses, such as IgG1, IgG4, IgD, CD8, or CD28 domains. The transmembrane domain can be derived from a variety of different T cell proteins, such as CD3ζ, CD4, CD8, CD28 or inducible T cell co-stimulator (ICOS). Several different endodomains have been used to generate CARs. For example, the endodomain can consist of a signaling chain having an ITAM, such as CD3ζ or FcεRIγ. In some instances, the endodomain further includes the intracellular portion of at least one additional co-stimulatory domain, such as CD28, 4-1BB (CD137, TNFRSF9), OX-40 (CD134), CD30, ICOS, CD27, MYD88-CD40, killer cell immunoglobulin-like receptor 2DS2 (KIR2DS2) and/or DAP10.

Immune cells (e.g., T cells, B cells, NK cells or macrophages) expressing CARs can be used to target a specific cell type, such as a cell expressing FasL. Thus, the monoclonal antibodies disclosed herein can be used to engineer immune cells that express a CAR containing the FasL-specific monoclonal antibody, thereby targeting the engineered immune cells to cells expressing FasL.

Multispecific (such as bispecific) or bicistronic CARs are also contemplated by the present disclosure. In some aspects, the multispecific or bispecific CAR includes an antibody specific for FasL and an antibody specific for a second protein. Similarly, a bicistronic CAR includes two CAR molecules expressed from the same construct where one CAR molecule is a FasL-targeted CAR and the second CAR targets a different protein. See, for example, Qin et al., Blood 130:810, 2017; and WO/2018/213337.

Accordingly, provided herein are CARs that include a FasL-specific antibody, such as any one of the antibodies disclosed herein. Also provided are isolated nucleic acid molecules and vectors encoding the CARs (including bispecific and bicistronic CARs), and host cells, such as immune cells (e.g., T cells, B cells, NK cells, or macrophages) expressing the CARs, bispecific CAR or bicistronic CARs. Immune cells expressing CARs comprised of a FasL-specific monoclonal antibody can be used for the treatment of diseases, disorders or conditions associated with Fas/FasL signaling.

Also provided herein are FasL-specific monoclonal antibodies modified to enable their use with a universal CAR system. Universal CAR systems increase CAR flexibility and expand their use to additional antigens. Currently, for each patient who receives CAR-T cell therapy, autologous T cells are cultured, expanded, and modified to express an antigen-specific CAR. This process is lengthy and expensive, limiting its use. Universal CARs are based on a system in which the signaling components of the CAR are split from the antigen-binding portion of the molecule but come together using a “lock-key” system. For example, biotin-binding immune receptor (BBIR) CARs are comprised of an intracellular T cell signaling domain fused to an extracellular domain comprising avidin. Biotinylated antigen-specific (such as FasL-specific) monoclonal antibodies can then bind the BBIR to direct immune cells to FasL-expressing cells. Another example is the split, universal and programmable (SUPRA) CAR system. In the SUPRA system, the CAR includes the intracellular signaling domains fused to an extracellular leucine zipper, which is paired with an antigen-specific monoclonal antibody fused to a cognate leucine zipper. For a review of universal CAR systems, see, for example, Zhao et al., J Hematol Oncol 11 (1): 132, 2018; and Cho et al., Cell 173:1426-1438, 2018. In some aspects herein, the FasL-specific antibody is fused to one component of a specific binding pair. In some examples, the antibody is fused to a leucine zipper or biotin.

Another type of universal CAR can be generated using a sortase enzyme. A sortase is a prokaryotic enzyme that modifies surface proteins by recognizing and cleaving a carboxyl-terminal sorting signal. Sortase catalyzes transpeptidation between a sortase recognition motif and a sortase acceptor motif. Thus, antigen-specific CARs can be generated by contacting an antigen-specific antibody fused to a sortase recognition motif with a portion of a CAR molecule that includes the intracellular signaling domain(s), a transmembrane region and an extracellular portion comprising a sortase acceptor motif. In the presence of the sortase enzyme, the two components become covalently attached to form a complete antigen-specific CAR. Accordingly, in some aspects herein, a FasL-specific antibody is modified to include a sortase recognition motif (see, for example, PCT Publication No. WO 2016/014553).

In some aspects, the FasL-specific CAR is expressed in allogeneic immune cells (such as T cells, B cells, NK cells, or macrophages), such as allogeneic immune cells from a healthy donor(s). In some examples, the allogeneic cells are genetically engineered to express the FasL-specific CAR, for example by disrupting expression of an endogenous T cell receptor by insertion of the CAR (see, for example, MacLeod et al., Mol Ther 25 (4): 949-961, 2017). Gene editing can be performed using any appropriate gene editing system, such as CRISPR/Cas9, zinc finger nucleases or transcription activator-like effector nucleases (TALEN).

### VII. Immunoconjugates

The disclosed monoclonal antibodies can be conjugated to a therapeutic agent or effector molecule. Immunoconjugates include, but are not limited to, molecules in which there is a covalent linkage of a therapeutic agent to an antibody. A therapeutic agent is an agent with a particular biological activity directed against a particular target molecule or a cell bearing a target molecule. A skilled person will appreciate that therapeutic agents can include various drugs such as vinblastine, daunomycin and the like, cytotoxins such as native or modified Pseudomonas exotoxin or diphtheria toxin, encapsulating agents (such as liposomes) that contain pharmacological compositions, radioactive agents such as ¹²⁵I, ³²P, ¹⁴C, ³H and ³⁵S, photon absorbers such as IR700, and other labels, target moieties and ligands.

The choice of a particular therapeutic agent depends on the particular target molecule or cell, and the desired biological effect. Thus, for example, the therapeutic agent can be a cytotoxin that is used to bring about the death of a particular target cell (such as a cancer cell). Conversely, where it is desired to invoke a non-lethal biological response, the therapeutic agent can be conjugated to a non-lethal pharmacological agent or a liposome containing a non-lethal pharmacological agent.

With the therapeutic agents and monoclonal antibodies described herein, one of skill can readily construct a variety of clones containing functionally equivalent nucleic acids, such as nucleic acids which differ in sequence but which encode the same effector moiety or antibody sequence. Thus, the present disclosure provides nucleic acids encoding antibodies and conjugates and fusion proteins thereof.

Effector molecules can be linked to a monoclonal antibody of interest using any number of means known to those of skill in the art. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers are well-known and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In general, a monoclonal antibody is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bispecific antibody or a diabody), a detection agent, a photon absorber, a pharmaceutical agent, and/or a protein or peptide that can mediate association of the antibody or antibody portion with another molecule (such as a streptavidin core region or a polyhistidine tag).

One type of derivatized antibody is produced by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

In some circumstances, it is desirable to free the effector molecule from the antibody when the immunoconjugate has reached its target site. Therefore, in these circumstances, immunoconjugates include linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

A monoclonal antibody provided herein can also be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP) and yellow fluorescent protein (YFP). An antibody can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin, and detected through indirect measurement of avidin or streptavidin binding. The avidin itself can also be conjugated with an enzyme or a fluorescent label.

An antibody provided herein may be labeled with a magnetic agent, such as gadolinium. Antibodies can also be labeled with lanthanides (such as europium and dysprosium), and manganese. Paramagnetic particles such as superparamagnetic iron oxide are also of use as labels. An antibody may also be labeled with a predetermined polypeptide epitope recognized by a secondary reporter (such as leucine zipper pair sequences, binding sites for secondary antibodies, metal binding domains, epitope tags). In some aspects, labels are attached by spacer arms of various lengths to reduce potential steric hindrance.

An antibody provided herein can also be labeled with a radiolabeled amino acid. The radiolabel may be used for both diagnostic and therapeutic purposes. For instance, the radiolabel may be used to detect expression of a target antigen by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I. In view of the large number of methods that have been reported for attaching a variety of radiodiagnostic compounds, radiotherapeutic compounds, labels (such as enzymes or fluorescent molecules), drugs, toxins, and other agents to antibodies, a skilled person can determine a suitable method for attaching a given agent to an antibody or other polypeptide.

An antibody disclosed herein can also be conjugated to a photon absorber. In some aspects, the photon absorber is a phthalocyanine dye, such as, but not limited to, IRDye® 700DX (also known as “IR700”). Antibody-photoabsorber conjugates can be used for photoimmunotherapy (for example to kill tumor cells).

An antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Toxins can be employed with the monoclonal antibodies described herein to produce immunotoxins. Exemplary toxins include ricin, abrin, diphtheria toxin and subunits thereof, as well as botulinum toxins A through F. These toxins are readily available from commercial sources (for example, Sigma Chemical Company, St. Louis, MO). Contemplated toxins also include variants of the toxins described herein (see, for example, see, U.S. Pat. Nos. 5,079,163 and 4,689,401). In one aspect, the toxin is Pseudomonas exotoxin (PE) (U.S. Pat. No. 5,602,095). As used herein “Pseudomonas exotoxin” refers to a full-length native (naturally occurring) PE or a PE that has been modified. Such modifications can include, but are not limited to, elimination of domain Ia, various amino acid deletions in domains Ib, II and III, single amino acid substitutions and the addition of one or more sequences at the carboxyl terminus (for example, see Siegall et al., J. Biol. Chem. 264:14256-14261, 1989).

PE employed with the monoclonal antibodies described herein can include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell. Cytotoxic fragments of PE include PE40, PE38, and PE35. For additional description of PE and variants thereof, see for example, U.S. Pat. Nos. 4,892,827; 5,512,658; 5,602,095; 5,608,039; 5,821,238; and 5,854,044; U.S. Patent Application Publication No. 2015/0099707; PCT Publication Nos. WO 99/51643, WO 2007/016150, WO 2009/032954, WO 2011/032022 and WO 2014/052064; Pai et al., Proc. Natl. Acad. Sci. USA 88:3358-3362, 1991; Kondo et al., J. Biol. Chem. 263:9470-9475, 1988; Pastan et al., Biochim. Biophys. Acta 1333: C1-C6, 1997; Weldon et al., Blood 113 (16): 3792-3800, 2009; and Onda et al., Proc Natl Acad Sci USA 105 (32): 11311-11316, 2008.

### VIII. Antibody-Drug Conjugates (ADCs)

ADCs are compounds comprised of an antigen-specific antibody (such as a FasL-specific antibody) and a drug, for example a cytotoxic agent (such as an anti-microtubule agent or cross-linking agent). Because ADCs are capable of specifically targeting cells expressing a particular antigen, the drug can be much more potent than agents used for standard systemic therapy. For example, the most common cytotoxic drugs currently used with ADCs have an IC₅₀ that is 100- to 1000-fold more potent than conventional chemotherapeutic agents. Exemplary cytotoxic drugs include anti-microtubule agents, such as maytansinoids and auristatins (such as auristatin E and auristatin F). Other cytotoxins for use with ADCs include pyrrolobenzodiazepines (PBDs), which covalently bind the minor groove of DNA to form interstrand crosslinks. In some instances, ADCs include a 1:2 to 1:4 ratio of antibody provided herein to drug (Bander, Clinical Advances in Hematology & Oncology 10 (8; suppl 10): 3-7, 2012).

The antibody and drug can be linked by a cleavable or non-cleavable linker. However, in some instances, it is desirable to have a linker that is stable in the circulation to prevent systemic release of the cytotoxic drug that could result in significant off-target toxicity. Non-cleavable linkers prevent release of the cytotoxic agent before the ADC is internalized by the target cell. Once in the lysosome, digestion of the antibody by lysosomal proteases results in the release of the cytotoxic agent (Bander, Clinical Advances in Hematology & Oncology 10 (8; suppl 10): 3-7, 2012).

One method for site-specific and stable conjugation of a drug to a monoclonal antibody (or an antibody-Fc fusion protein) is via glycan engineering. Monoclonal antibodies have one conserved N-linked oligosaccharide chain at the Asn297 residue in the CH2 domain of each heavy chain (Qasba et al., Biotechnol Prog 24:520-526, 2008). Using a mutant β1,4-galactosyltransferase enzyme (Y289L-Gal-T1; U.S. Patent Application Publication Nos. 2007/0258986 and 2006/0084162), 2-keto-galactose is transferred to free GlcNAc residues on the antibody heavy chain to provide a chemical handle for conjugation.

The oligosaccharide chain attached to monoclonal antibodies can be classified into three groups based on the terminal galactose residues-fully galactosylated (two galactose residues; IgG-G2), one galactose residue (IgG-G1) or completely degalactosylated (IgG-G0). Treatment of a monoclonal antibody with β1,4-galactosidase converts the antibody to the IgG-G0 glycoform. The mutant β1,4-galactosyltransferase enzyme can transfer 2-keto-galactose or 2-azido-galactose from their respective UDP derivatives to the GlcNAc residues on the IgG-G1 and IgG-G0 glycoforms. The chemical handle on the transferred sugar enables conjugation of a variety of molecules to the monoclonal antibody via the glycan residues (Qasba et al., Biotechnol Prog 24:520-526, 2008).

Provided herein are ADCs that include a drug (such as an anti-cancer agent) conjugated to a monoclonal antibody that specifically binds FasL. In some aspects, the drug is a small molecule. In some examples, the drug is a cross-linking agent, an anti-microtubule agent and/or anti-mitotic agent, or any cytotoxic agent suitable for mediating killing of tumor cells. Exemplary cytotoxic agents include, but are not limited to, a PBD, an auristatin, a maytansinoid, dolastatin, calicheamicin, nemorubicin and its derivatives, PNU-159682, anthracycline, vinca alkaloid, taxane, trichothecene, CC1065, camptothecin, elinafide, a combretastain, a dolastatin, a duocarmycin, an enediyne, a geldanamycin, an indolino-benzodiazepine dimer, a puromycin, a tubulysin, a hemiasterlin, a spliceostatin, or a pladienolide, as well as stereoisomers, isosteres, analogs, and derivatives thereof that have cytotoxic activity.

In some aspects, the ADC can further include a linker. In some examples, the linker is a bifunctional or multifunctional moiety that can be used to link one or more drug moieties to an antibody to form an ADC. In some aspects, ADCs are prepared using a linker having reactive functionalities for covalently attaching to the drug and to the antibody. For example, a cysteine thiol of an antibody can form a bond with a reactive functional group of a linker or a drug-linker intermediate to make an ADC.

In some examples, a linker has a functionality that is capable of reacting with a free cysteine present on an antibody to form a covalent bond. Exemplary linkers with such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates.

In some examples, a linker has a functionality that can react with an electrophilic group present on an antibody. Examples of such electrophilic groups include, but are not limited to, aldehyde and ketone carbonyl groups. In some cases, a heteroatom of the reactive functionality of the linker can react with an electrophilic group on an antibody and form a covalent bond to an antibody unit. Non-limiting examples include hydrazide, oxime, amino, hydrazine, thiosemicarbazone, hydrazine carboxylate and arylhydrazide.

In some examples, the linker is a cleavable linker, which facilitates release of the drug. Examples of cleavable linkers include acid-labile linkers (for example, comprising hydrazone), protease-sensitive linkers (for example, peptidase-sensitive), photolabile linkers, and disulfide-containing linkers (Chari et al., Cancer Res 52:127-131, 1992; U.S. Pat. No. 5,208,020).

The ADCs disclosed herein can be used for the treatment of a disease, disorder or condition associated with Fas/FasL signaling, alone or in combination with another therapeutic agent and/or in combination with any standard therapy for the treatment of a disease, disorder or condition associated with Fas/FasL signaling (for example, cancer, sepsis, myocardial infarction, stroke, hepatic ischemia-reperfusion injury, kidney ischemia-reperfusion injury, interstitial lung disease, autoimmune disease, or COVID-19).

### IX. Antibody-Nanoparticle Conjugates

The monoclonal antibodies disclosed herein can be conjugated to a variety of different types of nanoparticles to deliver cytotoxic agents or other therapeutic agents directly to FasL-expressing cells. The use of nanoparticles reduces off-target side effects and can also improve drug bioavailability and reduce the dose of a drug required to achieve a therapeutic effect. Nanoparticle formulations can be tailored to suit the drug that is to be carried or encapsulated within the nanoparticle. For example, hydrophobic molecules can be incorporated inside the core of a nanoparticle, while hydrophilic drugs can be carried within an aqueous core protected by a polymeric or lipid shell. Examples of nanoparticles include, but are not limited to, nanospheres, nanocapsules, liposomes, dendrimers, polymeric micelles, niosomes, and polymeric nanoparticles (Fay and Scott, Immunotherapy 3 (3): 381-394, 2011).

Liposomes are common types of nanoparticles used for drug delivery. An antibody conjugated to a liposome is often referred to as an “immunoliposome.” The liposomal component of an immunoliposome is typically a lipid vesicle of one or more concentric phospholipid bilayers. In some cases, the phospholipids are composed of a hydrophilic head group and two hydrophobic chains to enable encapsulation of both hydrophobic and hydrophilic drugs. Conventional liposomes are rapidly removed from the circulation via macrophages of the reticuloendothelial system (RES). To generate long-circulating liposomes, the composition, size and charge of the liposome can be modulated. The surface of the liposome may also be modified, such as with a glycolipid or sialic acid. For example, the inclusion of polyethylene glycol (PEG) significantly increases circulation half-life. Liposomes for use as drug delivery agents, including for preparation of immunoliposomes, have been described in the art (see, for example, Paszko and Senge, Curr Med Chem 19 (31) 5239-5277, 2012; Immordino et al., Int J Nanomedicine 1 (3): 297-315, 2006; U.S. Patent Application Publication Nos. 2011/0268655; 2010/00329981).

Niosomes are non-ionic surfactant-based vesicles having a structure similar to liposomes. The membranes of niosomes are composed only of nonionic surfactants, such as polyglyceryl-alkyl ethers or N-palmitoylglucosamine. Niosomes range from small, unilamellar to large, multilamellar particles. These nanoparticles are monodisperse, water-soluble, chemically stable, have low toxicity, are biodegradable and non-immunogenic, and increase bioavailability of encapsulated drugs.

Dendrimers include a range of branched polymer complexes. These nanoparticles are water-soluble, biocompatible and are sufficiently non-immunogenic for human use. Generally, dendrimers consist of an initiator core, surrounded by a layer of a selected polymer that is grafted to the core, forming a branched macromolecular complex. Dendrimers are typically produced using polymers such as poly(amidoamine) or poly(L-lysine). Dendrimers have been used for a variety of therapeutic and diagnostic applications, including for the delivery of DNA, RNA, bioimaging contrast agents, chemotherapeutic agents and other drugs.

Polymeric micelles are composed of aggregates of amphiphilic co-polymers (consisting of both hydrophilic and hydrophobic monomer units) assembled into hydrophobic cores, surrounded by a corona of hydrophilic polymeric chains exposed to the aqueous environment. In many cases, the polymers used to prepare polymeric micelles are heterobifunctional copolymers composed of a hydrophilic block of PEG, poly(vinyl pyrrolidone) and hydrophobic poly(L-lactide) or poly(L-lysine) that forms the particle core. Polymeric micelles can be used to carry drugs that have poor solubility. These nanoparticles have been used to encapsulate a number of drugs, including doxorubicin and camptothecin. Cationic micelles have also been developed to carry DNA or RNA molecules.

Polymeric nanoparticles include both nanospheres and nanocapsules. Nanospheres consist of a solid matrix of polymer, while nanocapsules contain an aqueous core. The formulation selected typically depends on the solubility of the therapeutic agent to be carried/encapsulated; poorly water-soluble drugs are more readily encapsulated within nanospheres, while water-soluble and labile drugs, such as DNA and proteins, are more readily encapsulated within nanocapsules. The polymers used to produce these nanoparticles include, for example, poly(acrylamide), poly(ester), poly(alkylcyanoacrylates), poly(lactic acid) (PLA), poly(glycolic acids) (PGA), and poly(D,L-lactic-co-glycolic acid) (PLGA).

Antibodies provided herein can be conjugated to a suitable nanoparticle according to standard methods known in the art. For example, conjugation can be either covalent or non-covalent. In some aspects in which the nanoparticle is a liposome, the antibody is attached to a sterically stabilized, long circulation liposome via a PEG chain. Coupling of antibodies or antibody fragments to a liposome can also involve thioester bonds, for example by reaction of thiols and maleimide groups. Cross-linking agents can be used to create sulfhydryl groups for attachment of antibodies to nanoparticles (Paszko and Senge, Curr Med Chem 19 (31) 5239-5277, 2012).

### X. Nucleic Acid Molecules

Nucleic acid molecules (for example, DNA, cDNA, mRNA, or RNA molecules) encoding the amino acid sequences of the disclosed monoclonal antibodies, fusion proteins, multi-specific antibodies, CARs, and immunoconjugates that specifically bind to FasL, are provided. Nucleic acid molecules encoding these molecules can readily be produced using the amino acid sequences provided herein (such as the CDR sequences), sequences available in the art (such as framework or constant region sequences), and the genetic code. In some aspects, the nucleic acid molecules can be expressed in a host cell (such as a mammalian cell, yeast cell or a bacterial cell) to produce a disclosed monoclonal antibody, fusion protein, multi-specific antibody, CAR or immunoconjugate.

The genetic code can be used to construct a variety of functionally equivalent nucleic acid sequences, such as nucleic acids that differ in their sequence, but which encode the same antibody sequence.

Nucleic acid molecules encoding the monoclonal antibodies, fusion proteins, multi-specific antibodies, CARs, and immunoconjugates that specifically bind to FasL can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4th ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as in bacterial, plant, yeast, insect, or mammalian cells. The monoclonal antibodies, fusion proteins, multi-specific antibodies, CARs, and immunoconjugates can be expressed as individual proteins including the monoclonal antibody (linked to an effector molecule or detectable marker as needed) or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011).

One or more DNA sequences encoding monoclonal antibodies, fusion proteins, multi-specific antibodies, CARs, and immunoconjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells, for example mammalian cells, such as the COS, CHO, HeLa and myeloma cell lines, can be used to express the disclosed antibodies. Methods of stable transfer, meaning that the foreign DNA is continuously maintained in the host may be used.

The expression of nucleic acids encoding the monoclonal antibodies, fusion proteins, multi-specific antibodies, CARs, and immunoconjugates described herein can be achieved by operably linking the DNA or cDNA to a promoter (which is either constitutive or inducible), followed by incorporation into an expression cassette. The promoter can be any promoter of interest, such as a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, expression cassettes can include, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding an antibody or conjugate described herein without diminishing its biological activity. Some modifications can be made to facilitate the cloning, expression, or incorporation of an antibody into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, fusion proteins, multi-specific antibodies and other conjugates can be purified according to standard procedures, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The monoclonal antibodies, fusion proteins, multi-specific antibodies, CARs, and immunoconjugates need not be 100% pure. Once purified, partially or to homogeneity as desired, if to be used prophylactically, the proteins should be substantially free of endotoxin.

### XI. Compositions

Compositions are provided that include one or more of the disclosed monoclonal antibodies that specifically bind FasL in a carrier. Compositions that include a fusion protein, multi-specific antibody, CAR, CAR-expressing cell, immunoconjugate, ADC, or antibody-nanoparticle conjugate are also provided, as are nucleic acid molecule and vectors encoding these molecules. The compositions can be prepared in unit dosage form for administration to a subject. The amount and timing of administration are at the discretion of the treating clinician to achieve the desired outcome. The monoclonal antibody, fusion protein, multi-specific antibody, CAR, CAR-expressing cell, immunoconjugate, ADC, antibody-nanoparticle conjugate, isolated nucleic acid molecule, vector or composition can be formulated for systemic or local administration. In one example, the composition is formulated for intravenous administration. In other examples, the composition is formulated for intramuscular administration or intraperitoneal administration. In other examples, the composition is formulated for intratumoral administration. In further examples, the composition is formulated for subcutaneous administration.

In some aspects, the composition includes more than one FasL-specific monoclonal antibody disclosed herein, such as 2 or 3 different antibodies (or multiple fusion proteins, multi-specific antibodies, CARs, CAR-expressing immune cells, immunoconjugates, ADCs, antibody-nanoparticle conjugates, isolated nucleic acid molecules or vectors). Kits are also provided that include one or more FasL-specific monoclonal antibodies disclosed herein, such as 2 or 3 different antibodies (or multiple fusion proteins, multi-specific antibodies, CARs, CAR-expressing immune cells, immunoconjugates, ADCs, antibody-nanoparticle conjugates, isolated nucleic acid molecules or vectors). Such kits can include one or more other therapeutic agents, such as those provided herein (e.g., other mAb, chemotherapeutic agent, or combinations thereof).

The compositions for administration can include a solution of the monoclonal antibody, fusion protein, multi-specific antibody, CAR, CAR-expressing cell, immunoconjugate, ADC, antibody-nanoparticle conjugate, isolated nucleic acid molecule and/or vector in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of monoclonal antibody, fusion protein, multi-specific antibody, CAR, CAR-expressing cell, immunoconjugate, ADC, antibody-nanoparticle conjugate, nucleic acid and/or vector in these formulations can vary, and can be selected based on fluid volumes, viscosities, body weight and the like in accordance with the particular mode of administration selected and the subject's needs.

An exemplary pharmaceutical composition for intravenous administration includes about 0.1 to 10 mg of antibody (or fusion protein, multi-specific antibody, and the like) per subject per day. Dosages from 0.1 up to about 100 mg per subject per day may be used, particularly if the agent is administered to a secluded site and not into the circulatory or lymph system, such as into a body cavity or into a lumen of an organ. In some aspects, the composition can be a liquid formulation including one or more antibodies in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml. Actual methods for preparing administrable compositions will be known or apparent to those skilled in the art and are described in more detail in such publications as Remington: The Science and Practice of Pharmacy, The University of the Sciences in Philadelphia, Editor, Lippincott, Williams, & Wilkins, Philadelphia, PA, 21st Edition (2005).

The compositions disclosed herein can also be administered by other routes, including via inhalation or orally, such as by oral administration of yeast or bacteria (e.g., Lactococcus lactis) engineered to express a disclosed antibody or conjugate (see, e.g., Vandenbroucke et al., Mucosal Immunol 3 (1): 49-56, 2010).

The disclosed compositions may be provided in lyophilized form and rehydrated with sterile water before administration, although they are also provided in sterile solutions of known concentration. The antibody solution can be added to an infusion bag containing 0.9% sodium chloride, USP, and in some cases administered at a dosage of from 0.5 to 15 mg/kg of body weight. Considerable experience is available in the art in the administration of antibody drugs, which have been marketed in the U.S. since the approval of RITUXAN™ in 1997. The disclosed compositions can be administered by slow infusion, rather than in an intravenous push or bolus. In one example, a higher loading dose is administered, with subsequent, maintenance doses being administered at a lower level.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include, for example, microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the antibody-based compositions disclosed herein. Various degradable and nondegradable polymeric matrices for use in controlled drug delivery are known (Langer, Accounts Chem. Res. 26:537-542, 1993). For example, the block copolymer, polaxamer 407, exists as a viscous yet mobile liquid at low temperatures but forms a semisolid gel at body temperature. Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### XII. Methods of Treatment

Also provided herein are methods of inhibiting Fas ligand in a subject in need thereof. In some aspects, the method includes administering to the subject a therapeutically effective amount of a monoclonal antibody, fusion protein, multi-specific antibody, CAR, CAR-expressing cell, immunoconjugate, ADC, antibody-nanoparticle conjugate, isolated nucleic acid molecule, vector or composition disclosed herein. In some aspects, the subject has cancer, sepsis, myocardial infarction, stroke, hepatic ischemia-reperfusion injury, kidney ischemia-reperfusion injury, interstitial lung disease, autoimmune disease, or COVID-19.

Further provided herein are methods of treating a disease, disorder or condition associated with Fas/FasL signaling, such as but not limited to, cancer, sepsis, myocardial infarction, stroke, hepatic ischemia-reperfusion injury, kidney ischemia-reperfusion injury, interstitial lung disease, autoimmune disease, myelodysplastic syndrome (MDS), or COVID-19, in a subject. In some aspects, the method includes administering to the subject a therapeutically effective amount of a monoclonal antibody, fusion protein, multi-specific antibody, CAR, CAR-expressing cell, immunoconjugate, ADC, antibody-nanoparticle conjugate, isolated nucleic acid molecule, vector or composition disclosed herein.

In some examples of the disclosed methods, the methods reduce Fas/FasL signaling by at least 10%, at least 20%, at least 30%, at least 50%, at least 50%, at least 75%, at least 90%, at least 95%, at least 98%, at least 99% or 100%, for example relative to Fas/FasL signaling prior to treatment.

In some examples, the methods increase survival of a subject, such as by at least 10%, at least 20%, at least 30%, at least 50%, at least 50%, at least 75%, at least 90%, at least 95%, at least 98%, at least 99% or 100%, for example compared to survival in the absence of treatment. In some examples, the methods increase the subject's survival time, such as by at least 3 months, at least 6 months, at least 9 months, at least 12 months, at least 18 months, at least 24 months, at last 36 months, at least 48 months, or at least 60 months, for example relative to the survival time in the absence of treatment.

In some examples, the methods reduce inflammation in a subject, such as by at least 10%, at least 20%, at least 30%, at least 50%, at least 50%, at least 75%, at least 90%, at least 95%, at least 98%, at least 99% or 100%, for example relative to inflammation prior to treatment.

In some examples in which the subject has cancer, the methods decrease the size, volume and/or weight of a tumor by at least 10%, at least 20%, at least 30%, at least 50%, at least 50%, at least 75%, at least 90%, at least 95%, at least 98%, at least 99% or 100%, for example relative to the size, volume and/or weight of the tumor prior to treatment. In some examples in which the subject has cancer, the methods decrease the size, volume and/or weight of a metastasis by at least 10%, at least 20%, at least 30%, at least 50%, at least 50%, at least 75%, at least 90%, at least 95%, at least 98%, at least 99% or 100%, for example relative to the size, volume and/or weight of the metastasis prior to treatment.

In some aspects, the subject is administered a second therapy or therapeutic agent. For example, a subject with cancer can be treated with a second anti-cancer therapy, such as chemotherapy, biological therapy (e.g., a different monoclonal antibody), radiation therapy, surgical excision, cryosurgery, laser therapy and/or administration of a checkpoint inhibitor.

Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g., anti-androgens) and anti-angiogenesis agents. Other anti-cancer treatments include radiation therapy and antibodies (e.g., mAbs) that specifically target cancer cells or other cells (e.g., anti-PD-1, anti-PD-L1, anti-CLTA4, anti-EGFR, or anti-VEGF). In one example, a cancer is treated by administering a polypeptide, antibody, fusion protein, CAR, CAR-expressing cell, immunoconjugate, ADC, multi-specific antibody, antibody-nanoparticle conjugate, or composition disclosed herein and one or more therapeutic mAbs, such as one or more of a PD-L1 antibody (e.g., durvalumab, KN035, cosibelimab, BMS-936559, BMS935559, MEDI-4736, MPDL-3280A, or MEDI-4737), anti-PD-1 antibody (e.g., pembrolizumab, cemiplimab, or nivolumab), anti-EGFR antibody (e.g., cetuximab or panitumumab), anti-VEGF antibody (e.g., bevacizumab or ramucizumab), or CLTA-4 antibody (e.g., ipilimumab or tremelimumab). In one example, a cancer is treated by administering a composition disclosed herein and one or more monoclonal antibodies, for example: 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nivolumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, tisotumab vedotin-tftv, Trastuzumab, Tremelimumab, Tucotuzumab celmoleukin, Veltuzumab, Volociximab, Votumumab, Zalutumumab, or combinations thereof.

### XIII. Additional Aspects

Aspect 1. A monoclonal antibody that specifically binds Fas ligand, comprising a variable heavy (VH) domain and a variable light (V_{L}) domain, wherein:
the VH domain comprises the complementarity determining region 1 (CDR1), CDR2 and CDR3 sequences of SEQ ID NO: 1, and the V_{L} domain comprises the CDR1, CDR2 and CDR3 sequences of SEQ ID NO: 2;
the VH domain comprises the CDR1, CDR2 and CDR3 sequences of SEQ ID NO: 3, and the V_{L} domain comprises the CDR1, CDR2 and CDR3 sequences of SEQ ID NO: 4; or
the VH domain comprises the CDR1, CDR2 and CDR3 sequences of SEQ ID NO: 5, and the V_{L} domain comprises the CDR1, CDR2 and CDR3 sequences of SEQ ID NO: 6.

Aspect 2. The monoclonal antibody of aspect 1, wherein:
the VH domain CDR1, CDR2 and CDR3 sequences respectively comprise residues 31-35, 50-66 and 99-108 of SEQ ID NO: 1; and
the V_{L} domain CDR1, CDR2 and CDR3 sequences respectively comprise residues 24-34, 50-56 and 89-96 of SEQ ID NO: 2.

Aspect 3. The monoclonal antibody of aspect 2, wherein:
the amino acid sequence of the VH domain is at least 90% identical to SEQ ID NO: 1 and comprises residues 31-35, 50-66 and 99-108 of SEQ ID NO: 1; and
the amino acid sequence of the V_{L} domain is at least 90% identical to SEQ ID NO: 2 and comprises residues 24-34, 50-56 and 89-96 of SEQ ID NO: 2.

Aspect 4. The monoclonal antibody of any one of aspects 1-3, wherein:
the amino acid sequence of the VH domain comprises or consists of SEQ ID NO: 1; and the amino acid sequence of the V_{L} domain comprises or consists of SEQ ID NO: 2.

Aspect 5. The monoclonal antibody of aspect 1, wherein:
the VH domain CDR1, CDR2 and CDR3 sequences respectively comprise residues 31-35, 50-66 and 99-108 of SEQ ID NO: 3; and
the V_{L} domain CDR1, CDR2 and CDR3 sequences respectively comprise residues 24-34, 50-56 and 89-96 of SEQ ID NO: 4.

Aspect 6. The monoclonal antibody of aspect 5, wherein:
the amino acid sequence of the VH domain is at least 90% identical to SEQ ID NO: 3 and comprises residues 31-35, 50-66 and 99-108 of SEQ ID NO: 3; and
the amino acid sequence of the V_{L} domain is at least 90% identical to SEQ ID NO: 4 and comprises residues 24-34, 50-56 and 89-96 of SEQ ID NO: 4.

Aspect 7. The monoclonal antibody of any one of aspects 1 and 5-6, wherein:
the amino acid sequence of the VH domain comprises or consists of SEQ ID NO: 3; and the amino acid sequence of the V_{L} domain comprises or consists of SEQ ID NO: 4.

Aspect 8. The monoclonal antibody of aspect 1, wherein:
the VH domain CDR1, CDR2 and CDR3 sequences respectively comprise residues 31-35, 50-66 and 99-107 of SEQ ID NO: 5; and
the V_{L} domain CDR1, CDR2 and CDR3 sequences respectively comprise residues 23-36, 52-58 and 91-99 of SEQ ID NO: 6.

Aspect 9. The monoclonal antibody of aspect 8, wherein:
the amino acid sequence of the VH domain is at least 90% identical to SEQ ID NO: 5 and comprises residues 31-35, 50-66 and 99-107 of SEQ ID NO: 5; and
the amino acid sequence of the V_{L} domain is at least 90% identical to SEQ ID NO: 6 and comprises residues 23-36, 52-58 and 91-99 of SEQ ID NO: 6.

Aspect 10. The monoclonal antibody of any one of aspects 1 and 8-9, wherein:
the amino acid sequence of the VH domain comprises or consists of SEQ ID NO: 5; and
the amino acid sequence of the V_{L} domain comprises or consists of SEQ ID NO: 6.

Aspect 11. The monoclonal antibody of any one of aspects 1-10, further comprising a heavy chain constant region and a light chain constant region.

Aspect 12. The monoclonal antibody of aspect 11, wherein the heavy chain constant region is a human IgG4 heavy chain constant region.

Aspect 13. The monoclonal antibody of aspect 12, wherein the amino acid sequence of the human IgG4 heavy chain constant region comprises or consists of SEQ ID NO: 7.

Aspect 14. The monoclonal antibody of any one of aspects 11-13, wherein the light chain constant region is a human kappa light chain constant region.

Aspect 15. The monoclonal antibody of aspect 14, wherein the amino acid sequence of the human kappa light chain constant region comprises or consists of SEQ ID NO: 8.

Aspect 16. The monoclonal antibody of any one of aspects 11-15, comprising a heavy chain and a light chain, wherein:
the amino acid sequence of the heavy chain comprises or consists of SEQ ID NO: 9 and the amino acid sequence of the light chain comprises or consists of SEQ ID NO: 10; or
the amino acid sequence of the heavy chain comprises or consists of SEQ ID NO: 11 and the amino acid sequence of the light chain comprises or consists of SEQ ID NO: 12.

Aspect 17. A monoclonal antibody that binds to the same epitope as a Fas ligand-specific monoclonal antibody comprising:
a heavy chain comprising the amino acid sequence of SEQ ID NO: 9; and
a light chain comprising the amino acid sequence of SEQ ID NO: 10.

Aspect 18. The monoclonal antibody of aspect 17, wherein the epitope is a conformational epitope spanning R144 to Y189 of Fas ligand set forth as SEQ ID NO: 17.

Aspect 19. The monoclonal antibody of any one of aspects 1-18, wherein the antibody is a human antibody or a humanized antibody.

Aspect 20. The monoclonal antibody of any one of aspects 1-18, wherein the antibody is a chimeric antibody.

Aspect 21. A fusion protein, comprising the monoclonal antibody of any one of aspects 1-20 and a heterologous protein.

Aspect 22. The fusion protein of aspect 21, wherein the heterologous protein is an Fc protein.

Aspect 23. A multi-specific antibody comprising the monoclonal antibody of any one of aspects 1-20 and at least one additional monoclonal antibody or antigen-binding fragment thereof.

Aspect 24. The multi-specific antibody of aspect 23, which is a bispecific antibody.

Aspect 25. A chimeric antigen receptor (CAR) comprising the monoclonal antibody of any one of aspects 1-20.

Aspect 26. An isolated cell expressing the CAR of aspect 25.

Aspect 27. The isolated cell of aspect 26, wherein the cell is an immune cell.

Aspect 28. An immunoconjugate comprising the monoclonal antibody of any one of aspects 1-20 and an effector molecule.

Aspect 29. The immunoconjugate of aspect 28, wherein the effector molecule is a toxin, a detectable label or a photon absorber.

Aspect 30. An antibody-drug conjugate (ADC) comprising a drug conjugated to the monoclonal antibody of any one of aspects 1-20.

Aspect 31. An antibody-nanoparticle conjugate, comprising a nanoparticle conjugated to the monoclonal antibody of any one of aspects 1-20.

Aspect 32. The antibody-nanoparticle conjugate of aspect 31, wherein the nanoparticle comprises a polymeric nanoparticle, nanosphere, nanocapsule, liposome, dendrimer, polymeric micelle, or niosome.

Aspect 33. A nucleic acid molecule encoding the monoclonal antibody of any one of aspects 1-20, the fusion protein of aspect 21 or aspect 22, the multi-specific antibody of aspect 23 or aspect 24, the CAR of aspect 25, or the immunoconjugate of aspect 28 or aspect 29.

Aspect 34. The nucleic acid molecule of aspect 33, operably linked to a promoter.

Aspect 35. A vector comprising the nucleic acid molecule of aspect 33 or aspect 34.

Aspect 36. An isolated host cell comprising the nucleic acid molecule of aspect 33 or aspect 34, or the vector of aspect 35.

Aspect 37. A composition, comprising a pharmaceutically acceptable carrier and the monoclonal antibody of any one of aspects 1-20, the fusion protein of aspect 21 or aspect 22, the multi-specific antibody of aspect 23 or aspect 24, the CAR of aspect 25, the isolated cell of any one of aspects 26, 27 and 36, the immunoconjugate of aspect 28 or aspect 29, the ADC of aspect 30, the antibody-nanoparticle conjugate of aspect 31 or aspect 32, the nucleic acid molecule of aspect 33 or aspect 34, or the vector of aspect 35.

Aspect 38. A method of inhibiting Fas ligand in a subject in need thereof, comprising administering to the subject a therapeutically effective amount of the monoclonal antibody of any one of aspects 1-20, the fusion protein of aspect 21 or aspect 22, the multi-specific antibody of aspect 23 or aspect 24, the CAR of aspect 25, the isolated cell of any one of aspects 26, 27 and 36, the immunoconjugate of aspect 28 or aspect 29, the ADC of aspect 30, the antibody-nanoparticle conjugate of aspect 31 or aspect 32, the nucleic acid molecule of aspect 33 or aspect 34, the vector of aspect 35, or the composition of aspect 37, thereby inhibiting Fas ligand in the subject.

Aspect 39. The method of aspect 38, wherein the subject has cancer, sepsis, myocardial infarction, stroke, hepatic ischemia-reperfusion injury, kidney ischemia-reperfusion injury, interstitial lung disease, autoimmune disease, myelodysplastic syndrome (MDS), or coronavirus disease 2019 (COVID-19).

Aspect 40. The method of aspect 39, wherein the cancer is glioblastoma multiforme or a myelodysplastic syndrome.

Aspect 41. A method of treating cancer, sepsis, myocardial infarction, stroke, hepatic ischemia-reperfusion injury, kidney ischemia-reperfusion injury, interstitial lung disease, autoimmune disease, myelodysplastic syndrome (MDS), or coronavirus disease 2019 (COVID-19) in a subject, comprising administering to the subject a therapeutically effective amount of the monoclonal antibody of any one of aspects 1-20, the fusion protein of aspect 21 or aspect 22, the multi-specific antibody of aspect 23 or aspect 24, the CAR of aspect 25, the isolated cell of any one of aspects 26, 27 and 36, the immunoconjugate of aspect 28 or aspect 29, the ADC of aspect 30, the antibody-nanoparticle conjugate of aspect 31 or aspect 32, the nucleic acid molecule of aspect 33 or aspect 34, the vector of aspect 35, or the composition of aspect 37.

Aspect 42. The method of aspect 41, wherein the cancer is glioblastoma multiforme or a myelodysplastic syndrome.

The following examples are provided to illustrate certain particular features and/or aspects. These examples should not be construed to limit the disclosure to the particular features or aspects described.

### EXAMPLES

### Example 1: Monoclonal Antibodies Specific for Fas Ligand

This example describes three monoclonal antibodies that bind and block the function of human FasL. Two of the antibodies (M3T01 and M3T02) are human monoclonal antibodies isolated from human FasL-immunized transgenic mice with human antibody variable region genes (ATX-Gx™, Alloy Therapeutics). The third antibody is a mouse antibody (M3T03) selected from human FasL-immunized Balb/c mice. The studies described below demonstrate that M3T01 binds soluble and cell-surface FasL with high affinity, exhibits cross-reactivity with FasL from several different species, potently inhibits FasL-mediated apoptosis, and is highly stable.

### Antibody Sequences

M3T01 and M3T02 are fully human IgG4/Kappa monoclonal antibodies with an S228P alteration to prevent Fab arm exchange (see heavy chain constant region sequence set forth as SEQ ID NO: 7). The VH and V_{L} domain of M3T01 are set forth herein as SEQ ID NOs: 1 and 2, respectively; and the heavy chain and light chain of M3T01 are set forth herein as SEQ ID NOs: 9 and 10, respectively. The VH and V_{L} domain of M3T02 are set forth herein as SEQ ID NOs: 3 and 4, respectively; and the heavy chain and light chain of M3T02 are set forth herein as SEQ ID NOs: 11 and 12, respectively. M3T03 is a fully mouse antibody. The VH and V_{L} domain sequences are set forth herein as SEQ ID NOs: 5 and 6, respectively.

### Binding Assays

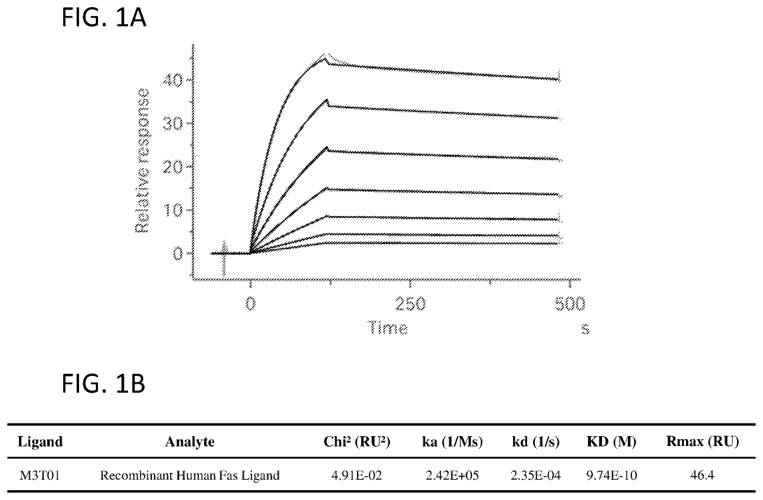

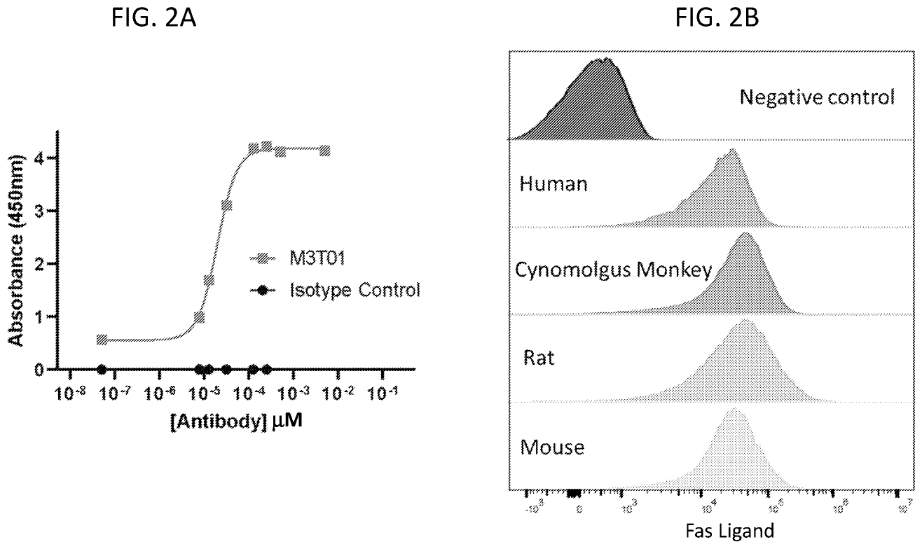

Binding of M3T01 to human FasL was measured by multi-dose SPR using BIACORE™ 8K. As shown in FIGS. 1A-1B, M3T01 bound to human FasL with high affinity (974 pM). In another study, binding of M3T01 to soluble FasL (sFasL) was measured by ELISA. The results show that M3T01 bound to sFasL with high affinity (FIG. 2A).

Next, assays were performed to evaluate binding of M3T01 to cell-surface, membrane-bound FasL from a variety of different species. HEK293T cells were transfected with vectors expressing either mouse, human, rat or cynomolgus macaque FasL and binding by M3T01 was measured by flow cytometry. As shown in FIG. 2B, M3T01 bound to cell-surface FasL from all species tested. These data demonstrate that M3T01 has broad species cross-reactivity.

Additional studies demonstrated that M3T01 binds cell surface FasL on activated human lymphocytes. In this study, human peripheral blood mononuclear cells (PBMCs) were activated with PMA/ionomycin and evaluated for FasL expression by western blot, qPCR and flow cytometry using M3T01 and NOK1 (a commercially available anti-human FasL antibody used as a positive control).

### FasL Epitope Bound by M3T01

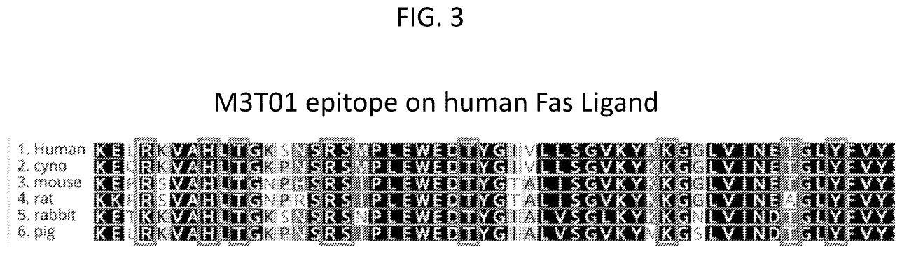

The epitope for M3T01 was characterized by CovalX. The methodology included High-Mass MALDI mass spectrometry analysis of M3T01/human FasL complexes after chemical cross-linking and protease digestion. The results demonstrate that M3T01 binds a conformational epitope on FasL that spans from R144 through Y189 (see FIG. 3; amino acid numbering based on human FasL set forth as SEQ ID NO: 17). The epitope of M3T01 is a highly conserved region of FasL across various species including cynomolgus monkey, rat, mouse, rabbit, and pig (FIG. 3).

### Functional Activity of M3T01

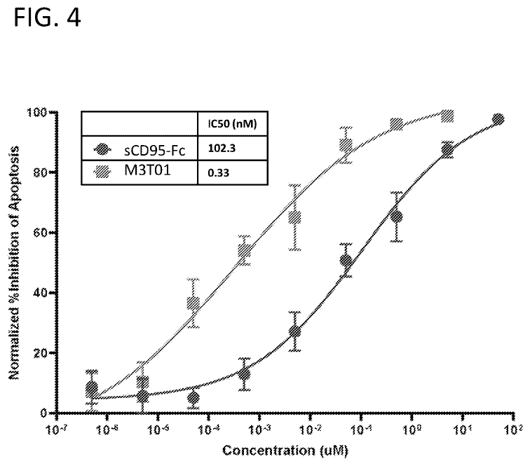

To evaluate functional activity of M3T01, an HEK293T cell line transfected to express high levels of human FasL (hFasL/HEK293T) was used to measure inhibition of apoptosis. This cell line is used to induce apoptosis in Jurkat target cells, which express the Fas receptor. Co-culture of these cell lines for 4 hours results in apoptosis of the Jurkat cells. To evaluate inhibition of FasL-mediated apoptosis, M3T01 antibody or a soluble CD95-Fc (sCD95-Fc) fusion protein targeting FasL was added to the hFasL/HEK293T cells prior to co-culture with Jurkat cells. As shown in FIG. 4, M3T01 exhibited greater potency for inhibiting apoptosis relative to sCD95-Fc. Specifically, this study demonstrated that M3T01 has an IC₅₀ of 0.33 nM, which is 310-fold more potent than sCD95-Fc (102.3 nM).

### Stability of M3T01

To evaluate stability of M3T01 under high stress conditions, M3T01 was incubated at 37° C. for 3 weeks and then tested for binding to cell surface FasL as well as inhibition of FasL-mediated apoptosis. Incubation of M3T01 at 37° C. for 3 weeks did not impact cell surface FasL binding or inhibition of FasL-mediated apoptosis. Similarly, incubation at 40° C. for 2 weeks resulted in no loss of M3T01 activity. Additionally, M3T01 was subjected to repeated freeze-thaw cycles (5 cycles of freezing at −80° C. followed by thawing at room temperature). Repeated freeze-thaw cycling did not reduce cell surface FasL binding or inhibition of FasL-mediated apoptosis.

### Tissue Cross-Reactivity

An extensive tissue cross-reactivity study was performed. The study evaluated 37 human tissues from 3 separate donors. This study showed excellent specificity, with M3T01 showing binding only to mononuclear cells in lymph nodes and reticulo-endothelial cells in the spleen (consistent with the known physiologic tissue expression of FasL). No binding of M3T01 was detected in other normal/healthy organs/tissues.

M3T01 also binds to fresh frozen human tumor specimens. Since M3T01 recognizes a conformational epitope present only in FasL in its native (non-denatured) state, it does not bind to FasL in formalin fixed paraffin embedded tissue specimens.

### Example 2: Variants of M3T01

Several variants of the M3T01 antibody were isolated and tested for binding affinity to human FasL using a single-dose SPR affinity measurement. Each variant clone includes the same CDR sequences as M3T01 but includes one or more amino acid substitutions in the framework region (FR) of the VH domain and/or VL domain. The variant sequences, substitutions relative to the M3T01 VH and VL domains, and measured binding affinity are listed in Table 2. The binding affinities reported in Table 2 are from a single-dose SPR study. The results demonstrate that binding affinity of each variant is not significantly different from M3T01 (represented by M27.2 in Table 2).

TABLE 2
Mutations in FR regions and their impact on binding affinity

| Clone | FR mutations | VH | VL | KD (M) |
|---|---|---|---|---|
| M27.1 | VH: N77D, | QVQLVESGGGVVQPGRSLRLSC | DIQMTQSPSTLSASVGDRVT | 1.36E−09 |
| | Q82E, V93M | AASGFTFSTYGIHWVRQAPGKG | ITCRASQSISSWLAWYQQKP | |
| | VL: L46F | LEWVAVIWYDGSDKFYADSVK | GKAPKFLIYQASSLESGVPS | |
| | | GRFTISRDNSKDTLYLEMNSLRA | RFSGSGSGTEFTLTISSLQPD | |
| | | EDTAMYYCARDRDNWNYFDY | DFATYYCQQYNSYITFGQG | |
| | | WGQGTLVTVSS (SEQ ID NO: 13) | TRLEIK (SEQ ID NO: 2) | |
| M27.2 | VH: none | QVQLVESGGGVVQPGRSLRLSC | DIQMTQSPSTLSASVGDRVT | 3.20E−09 |
| | VL: L46F | AASGFTFSTYGIHWVRQAPGKG | ITCRASQSISSWLAWYQQKP | |
| | | LEWVAVIWYDGSDKFYADSVK | GKAPKFLIYQASSLESGVPS | |
| | | GRFTISRDNSKNTLYLQMNSLRA | RFSGSGSGTEFTLTISSLQPD | |
| | | EDTAVYYCARDRDNWNYFDY | DFATYYCQQYNSYITFGQG | |
| | | WGQGTLVTVSS (SEQ ID NO: 1) | TRLEIK (SEQ ID NO: 2) | |
| M27.3 | VH: N77D, | QVQLVESGGGVVQPGRSLRLSC | DIQMTQSPSTLSASVGDRVT | 1.96E−09 |
| | Q82E, V93M | AASGFTFSTYGIHWVRQAPGKG | ITCRASQSISSWLAWYQQKP | |
| | VL: none | LEWVAVIWYDGSDKFYADSVK | GKAPKLLIYQASSLESGVPS | |
| | | GRFTISRDNSKDTLYLEMNSLRA | RFSGSGSGTEFTLTISSLQPD | |
| | | EDTAMYYCARDRDNWNYFDY | DFATYYCQQYNSYITFGQG | |
| | | WGQGTLVTVSS (SEQ ID NO: 13) | TRLEIK (SEQ ID NO: 16) | |
| M27.4 | VH: none | QVQLVESGGGVVQPGRSLRLSC | DIQMTQSPSTLSASVGDRVT | 3.10E−09 |
| | VL: none | AASGFTFSTYGIHWVRQAPGKG | ITCRASQSISSWLAWYQQKP | |
| | | LEWVAVIWYDGSDKFYADSVK | GKAPKLLIYQASSLESGVPS | |
| | | GRFTISRDNSKNTLYLQMNSLRA | RFSGSGSGTEFTLTISSLQPD | |
| | | EDTAVYYCARDRDNWNYFDY | DFATYYCQQYNSYITFGQG | |
| | | WGQGTLVTVSS (SEQ ID NO: 1) | TRLEIK (SEQ ID NO: 16) | |
| M27.5 | VH: N77D, | QVQLVESGGGVVQPGRSLRLSC | DIQMTQSPSTLSASVGDRVT | 3.44E−09 |
| | V93M | AASGFTFSTYGIHWVRQAPGKG | ITCRASQSISSWLAWYQQKP | |
| | VL: none | LEWVAVIWYDGSDKFYADSVK | GKAPKLLIYQASSLESGVPS | |
| | | GRFTISRDNSKDTLYLQMNSLRA | RFSGSGSGTEFTLTISSLQPD | |
| | | EDTAMYYCARDRDNWNYFDY | DFATYYCQQYNSYITFGQG | |
| | | WGQGTLVTVSS (SEQ ID NO: 14) | TRLEIK (SEQ ID NO: 16) | |
| M27.6 | VH: N77D, | QVQLVESGGGVVQPGRSLRLSC | DIQMTQSPSTLSASVGDRVT | 2.12E−09 |
| | Q82E | AASGFTFSTYGIHWVRQAPGKG | ITCRASQSISSWLAWYQQKP | |
| | VL: none | LEWVAVIWYDGSDKFYADSVK | GKAPKLLIYQASSLESGVPS | |
| | | GRFTISRDNSKDTLYLEMNSLRA | RFSGSGSGTEFTLTISSLQPD | |
| | | EDTAVYYCARDRDNWNYFDY | DFATYYCQQYNSYITFGQG | |
| | | WGQGTLVTVSS (SEQ ID NO: 15) | TRLEIK (SEQ ID NO: 16) | |

### Example 3: Clinical Testing of M3T01

The example describes evaluation of M3T01 in a phase I dose-escalation clinical trial in patients with advanced cancers that are refractory to standard therapies. Various drug dosages and treatment intervals are evaluated in the clinical trial. After the phase I clinical trial has established a safe/tolerable dose and treatment interval, phase II clinical trials are conducted in patients with sepsis, severe COVID-19 illness, and myocardial ischemia.

In view of the many possible aspects to which the principles of the disclosed subject matter may be applied, it should be recognized that the illustrated aspects are only examples of the disclosure and should not be taken as limiting the scope of the disclosure. Rather, the scope of the disclosure is defined by the following claims. We therefore claim all that comes within the scope and spirit of these claims.

1. A monoclonal antibody that specifically binds Fas ligand, comprising a variable heavy (VH) domain and a variable light (VL) domain, wherein:
the VH domain comprises the complementarity determining region 1 (CDR1), CDR2 and CDR3 sequences of SEQ ID NO: 1, and the VL domain comprises the CDR1, CDR2 and CDR3 sequences of SEQ ID NO: 2;
the VH domain comprises the CDR1, CDR2 and CDR3 sequences of SEQ ID NO: 3, and the VL domain comprises the CDR1, CDR2 and CDR3 sequences of SEQ ID NO: 4; or
the VH domain comprises the CDR1, CDR2 and CDR3 sequences of SEQ ID NO: 5, and the VL domain comprises the CDR1, CDR2 and CDR3 sequences of SEQ ID NO: 6.

2. The monoclonal antibody of claim 1, wherein:
the VH domain CDR1, CDR2 and CDR3 sequences respectively comprise residues 31-35, 50-66 and 99-108 of SEQ ID NO: 1; and the VL domain CDR1, CDR2 and CDR3 sequences respectively comprise residues 24-34, 50-56 and 89-96 of SEQ ID NO: 2;
the VH domain CDR1, CDR2 and CDR3 sequences respectively comprise residues 31-35, 50-66 and 99-108 of SEQ ID NO: 3; and the VL domain CDR1, CDR2 and CDR3 sequences respectively comprise residues 24-34, 50-56 and 89-96 of SEQ ID NO: 4; or
the VH domain CDR1, CDR2 and CDR3 sequences respectively comprise residues 31-35, 50-66 and 99-107 of SEQ ID NO: 5; and the VL domain CDR1, CDR2 and CDR3 sequences respectively comprise residues 23-36, 52-58 and 91-99 of SEQ ID NO: 6.

3. The monoclonal antibody of claim 2, wherein:
the amino acid sequence of the VH domain is at least 90% identical to SEQ ID NO: 1 and comprises residues 31-35, 50-66 and 99-108 of SEQ ID NO: 1; and the amino acid sequence of the VL domain is at least 90% identical to SEQ ID NO: 2 and comprises residues 24-34, 50-56 and 89-96 of SEQ ID NO: 2;
the amino acid sequence of the VH domain is at least 90% identical to SEQ ID NO: 3 and comprises residues 31-35, 50-66 and 99-108 of SEQ ID NO: 3; and the amino acid sequence of the VL domain is at least 90% identical to SEQ ID NO: 4 and comprises residues 24-34, 50-56 and 89-96 of SEQ ID NO: 4; or
the amino acid sequence of the VH domain is at least 90% identical to SEQ ID NO: 5 and comprises residues 31-35, 50-66 and 99-107 of SEQ ID NO: 5; and the amino acid sequence of the VL domain is at least 90% identical to SEQ ID NO: 6 and comprises residues 23-36, 52-58 and 91-99 of SEQ ID NO: 6.

4. The monoclonal antibody of claim 1, wherein:
the amino acid sequence of the VH domain comprises or consists of SEQ ID NO: 1 and the amino acid sequence of the VL domain comprises or consists of SEQ ID NO: 2;
the amino acid sequence of the VH domain comprises or consists of SEQ ID NO: 3 and the amino acid sequence of the VL domain comprises or consists of SEQ ID NO: 4; or
the amino acid sequence of the VH domain comprises or consists of SEQ ID NO: 5 and the amino acid sequence of the VL domain comprises or consists of SEQ ID NO: 6.

5. The monoclonal antibody of claim 1, further comprising a heavy chain constant region and a light chain constant region.

6. The monoclonal antibody of claim 5, wherein the heavy chain constant region is a human IgG4 heavy chain constant region and/or the light chain constant region is a human kappa light chain constant region.

7. The monoclonal antibody of claim 6, wherein the amino acid sequence of the human IgG4 heavy chain constant region comprises or consists of SEQ ID NO: 7 and/or the amino acid sequence of the human kappa light chain constant region comprises or consists of SEQ ID NO: 8.

8. The monoclonal antibody of claim 1, comprising a heavy chain and a light chain, wherein:
the amino acid sequence of the heavy chain comprises or consists of SEQ ID NO: 9 and the amino acid sequence of the light chain comprises or consists of SEQ ID NO: 10; or
the amino acid sequence of the heavy chain comprises or consists of SEQ ID NO: 11 and the amino acid sequence of the light chain comprises or consists of SEQ ID NO: 12.

9. A monoclonal antibody that binds to the same epitope as a Fas ligand-specific monoclonal antibody comprising:
a heavy chain comprising the amino acid sequence of SEQ ID NO: 9; and
a light chain comprising the amino acid sequence of SEQ ID NO: 10.

10. The monoclonal antibody of claim 9, wherein the epitope is a conformational epitope spanning R144 to Y189 of Fas ligand set forth as SEQ ID NO: 17.

11. The monoclonal antibody of claim 1, wherein the antibody is a human antibody, a humanized antibody, or a chimeric antibody.

12. A fusion protein, comprising the monoclonal antibody of claim 1 and a heterologous protein.

13. (canceled)

14. A multi-specific antibody comprising the monoclonal antibody of claim 1 and at least one additional monoclonal antibody or antigen-binding fragment thereof.

15. (canceled)

16. A chimeric antigen receptor (CAR) comprising the monoclonal antibody of claim 1.

17. An isolated cell expressing the CAR of claim 16.

18. (canceled)

19. An immunoconjugate comprising the monoclonal antibody of claim 1 and an effector molecule.

20. (canceled)

21. An antibody-drug conjugate (ADC) comprising a drug conjugated to the monoclonal antibody of claim 1.

22. An antibody-nanoparticle conjugate, comprising a nanoparticle conjugated to the monoclonal antibody of claim 1.

23. (canceled)

24. A nucleic acid molecule encoding the monoclonal antibody of claim 1.

25. The nucleic acid molecule of claim 24, operably linked to a promoter.

26. A vector comprising the nucleic acid molecule of claim 25.

27. An isolated host cell comprising the vector of claim 26.

28. A composition, comprising a pharmaceutically acceptable carrier and the monoclonal antibody of claim 1.

29. A method of inhibiting Fas ligand in a subject in need thereof, comprising administering to the subject a therapeutically effective amount of the monoclonal antibody of claim 1, thereby inhibiting Fas ligand in the subject.

30.-31. (canceled)

32. A method of treating cancer, sepsis, myocardial infarction, stroke, hepatic ischemia-reperfusion injury, kidney ischemia-reperfusion injury, interstitial lung disease, autoimmune disease, myelodysplastic syndrome (MDS), or coronavirus disease 2019 (COVID-19) in a subject, comprising administering to the subject a therapeutically effective amount of the monoclonal antibody of claim 1.

33. The method of claim 32, wherein the cancer is glioblastoma multiforme or a myelodysplastic syndrome.


SEQUENCE LISTING (22 sequences)

SEQ ID NO: 1 - protein, 119 aa, synthetic construct
QVQLVESGGGVVQPGRSLRLSCAASGFTFSTYGIHWVRQAPGKGLEWVAVIWYDGSDKFYADSVKGRFTISRDNSKNTLYLQMNSLRAEDTAVYYCARDRDNWNYFDYWGQGTLVTVSS

SEQ ID NO: 2 - protein, 106 aa, synthetic construct
DIQMTQSPSTLSASVGDRVTITCRASQSISSWLAWYQQKPGKAPKFLIYQASSLESGVPSRFSGSGSGTEFTLTISSLQPDDFATYYCQQYNSYITFGQGTRLEIK

SEQ ID NO: 3 - protein, 119 aa, synthetic construct
QVQLVDSGGGVVQPGRSLRLSCAASGFTFSSYGMHWVRQAPGKGPEWVAVIWYDGSNKYYADSVKGRFTISRDNSKNTLYLQMNSLRAEDTAVYYCARDRDNWNHFDYWGQGTLVTVSS

SEQ ID NO: 4 - protein, 106 aa, synthetic construct
EIVMTQSPATLSVSPGERATLSCRASQSFSSNLAWYQQKPGQAPRLLIYGASTRATGIPARFSGSGSGTEFTLTISSLQSEDFAVYYCQQYNNWLTFGGGTKVEIK

SEQ ID NO: 5 - protein, 118 aa, synthetic construct
QIQLVQSGPDLKKPGETVKISCKASGYTFTNYGMNWVKKAPGKGLKWMGWINTNTGEPSYAEEFKGRFAFSLETSAGTAYLHINNLKNEDTATYFCVKYSRYYAMDFWGQGTSVTVSS

SEQ ID NO: 6 - protein, 109 aa, synthetic construct
QAVVTQESALTTSPGETVTLTCRSSTGAVTTSNYANWVQEKPDHLFTGLIGDTNNRAPGVPARFSGSLIGDKAALTITGAQTEDEAMYFCALWYSNHWVFGGGTKLTVL

SEQ ID NO: 7 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 107 aa, Homo sapiens [sequence omitted]

SEQ ID NO: 9 - protein, 446 aa, synthetic construct
QVQLVESGGGVVQPGRSLRLSCAASGFTFSTYGIHWVRQAPGKGLEWVAVIWYDGSDKFYADSVKGRFTISRDNSKNTLYLQMNSLRAEDTAVYYCARDRDNWNYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 213 aa, synthetic construct
DIQMTQSPSTLSASVGDRVTITCRASQSISSWLAWYQQKPGKAPKFLIYQASSLESGVPSRFSGSGSGTEFTLTISSLQPDDFATYYCQQYNSYITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, synthetic construct
QVQLVDSGGGVVQPGRSLRLSCAASGFTFSSYGMHWVRQAPGKGPEWVAVIWYDGSNKYYADSVKGRFTISRDNSKNTLYLQMNSLRAEDTAVYYCARDRDNWNHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 213 aa, synthetic construct
EIVMTQSPATLSVSPGERATLSCRASQSFSSNLAWYQQKPGQAPRLLIYGASTRATGIPARFSGSGSGTEFTLTISSLQSEDFAVYYCQQYNNWLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
QVQLVESGGGVVQPGRSLRLSCAASGFTFSTYGIHWVRQAPGKGLEWVAVIWYDGSDKFYADSVKGRFTISRDNSKDTLYLEMNSLRAEDTAMYYCARDRDNWNYFDYWGQGTLVTVSS

SEQ ID NO: 14 - protein, 119 aa, synthetic construct
QVQLVESGGGVVQPGRSLRLSCAASGFTFSTYGIHWVRQAPGKGLEWVAVIWYDGSDKFYADSVKGRFTISRDNSKDTLYLQMNSLRAEDTAMYYCARDRDNWNYFDYWGQGTLVTVSS

SEQ ID NO: 15 - protein, 119 aa, synthetic construct
QVQLVESGGGVVQPGRSLRLSCAASGFTFSTYGIHWVRQAPGKGLEWVAVIWYDGSDKFYADSVKGRFTISRDNSKDTLYLEMNSLRAEDTAVYYCARDRDNWNYFDYWGQGTLVTVSS

SEQ ID NO: 16 - protein, 106 aa, synthetic construct
DIQMTQSPSTLSASVGDRVTITCRASQSISSWLAWYQQKPGKAPKLLIYQASSLESGVPSRFSGSGSGTEFTLTISSLQPDDFATYYCQQYNSYITFGQGTRLEIK

SEQ ID NO: 17 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 18 - protein, 280 aa, Macaca fascicularis
MQQPFNYPYPQIYWVDSSASSPWAPPGTVLPCPTSVPRRPGQRRPPPPPPPPPLPPPPPSPLPPLPLPPLKKRGNHSTGLCLLVMFFMVLVALVGLGLGMFQLFHLQKELAELRESTSQKHTASSLEKQIGHPSPPPEKKEQRKVAHLTGKPNSRSMPLEWEDTYGIVLLSGVKYKKGGLVINETGLYFVYSKVYFRGQSCTNLPLSHKVYMRNSKYPQDLVMMEGKMMSYCTTGQMWAHSSYLGAVFNLTSADHLYVNVSELSLVNFEESQTFFGLYKL

SEQ ID NO: 19 - protein, 279 aa, Mus musculus
MQQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSYLGAVFNLTSADHLYVNISQLSLINFEESKTFFGLYKL

SEQ ID NO: 20 - protein, 278 aa, Rattus norvegicus
MQQPVNYPCPQIYWVDSSATSPWAPPGSVFSCPSSGPRGPGQRRPPPPPPPPSPLPPPSQPPPLPPLSPLKKKDNIELWLPVIFFMVLVALVGMGLGMYQLFHLQKELAELREFTNHSLRVSSFEKQIANPSTPSETKKPRSVAHLTGNPRSRSIPLEWEDTYGTALISGVKYKKGGLVINEAGLYFVYSKVYFRGQSCNSQPLSHKVYMRNFKYPGDLVLMEEKKLNYCTTGQIWAHSSYLGAVFNLTVADHLYVNISQLSLINFEESKTFFGLYKL

SEQ ID NO: 21 - protein, 285 aa, Oryctolagus cuniculus
MQQPFSYPYPQIYWVDSTASSPWAPPGSVLPCPSSVPERPGQRRPPPPLPPPPPPLPPPPLPPLPPLPPLPPPPLKKRKDHSTGLCLLLMFFMVLVAXVGLGWDVQLYHLQXELAELRESFSQRHTASSSMEKQTAHPSPPQEKKETKKVAHLTGKSNSRSNPLEWEDTYGIALVSGLKYKKGNLVINDTGLYFVYSKVYFRGQSCSNQPLTHKVYMKNSKYHQDLMLMEEKMMNYCTTGQMWARSSYLGAVFNLTSADHVYVNVSEISLVNFEESKTFFGLYKL

SEQ ID NO: 22 - protein, 282 aa, Sus scrofa
MQQPFNYPYPQIFWVDSSATSPWASPGSVFPCPASVPGRPGQRRPPPPPPPPPPPPTLLPSRPLPPLPPPSLKKKRDHNAGLCLLVMFFMVLVALVGLGLGMFQLFHLQKELTELRESASQRHTESSLEKQIGHPNLPSEKKELRKVAHLTGKPNSRSIPLEWEDTYGIALVSGVKYMKGSLVINDTGLYFVYSKVYFRGQYCNNQPLSHKVYTRNSRYPQDLVLMEGKMMNYCTTGQMWARSSYLGAVFNLTSADHLYVNVSELSLVNFEESKTFFGLYKL